(19) United States
(12) Patent Application Publication
(10) Pub. No.: US 2014/0056953 A1
(43) Pub. Date: Feb. 27, 2014

(54) FATTY ACID ACYLATED AMINO ACIDS FOR ORAL PEPTIDE DELIVERY

(71) Applicants: Florian Anders Foeger, Malmo (SE); Abdallah Makhlof, Bagsvaerd (DK); Herbert Hoyer, Munich (DE)

(72) Inventors: Florian Anders Foeger, Malmo (SE); Abdallah Makhlof, Bagsvaerd (DK); Herbert Hoyer, Munich (DE)

(73) Assignee: NOVO NORDISK A/S, Bagsvaerd (DK)

(21) Appl. No.: 14/111,274

(22) PCT Filed: Apr. 12, 2012
(86) PCT No.: PCT/EP12/56708

§ 371 Date: Nov. 12, 2013

Related U.S. Application Data
(60) Provisional application No. 61/477,719, filed on Apr. 21, 2011.

(30) Foreign Application Priority Data
Apr. 14, 2011 (EP) 11162411.0

(51) Int. Cl. A61K 47/18; A61K 38/28

(52) U.S. Cl. CPC A61K 47/183; A61K 38/28
USPC 424/400; 514/5.9

(57) ABSTRACT

The present invention relates to fatty acid acylated amino acids (FA-aa's) acting as permeation enhancers for oral delivery of therapeutic macromolecules such as peptides and pharmaceutical compositions comprising such FA-aa's.

### TECHNICAL FIELD

The technical field of this invention relates to fatty acid acylated amino acids (FA-aa's) for oral delivery of therapeutic hydropilic peptides and proteins and pharmaceutical compositions comprising such FA-aa's.

### BACKGROUND

Many pathological states due to deficiencies in or complete failure of the production of a certain macromolecules (e.g. proteins and peptides) are treated with an invasive and inconvenient parenteral administration of therapeutic macromolecules, such as hydrophilic peptides or proteins. One example hereof is the administration of insulin in the treatment of insulin dependent patients, who are in need of one or more daily doses of insulin. The oral route is desirable for administration due to its non-invasive nature and has a great potential to decrease the patient's discomfort related to drug administration and to increase drug compliance. However several barriers exist; such as the enzymatic degradation in the gastrointestinal (GI) tract, drug efflux pumps, insufficient and variable absorption from the intestinal mucosa, as well as first pass metabolism in the liver and until now no products for oral delivery of therapeutic hydrophilic proteins are found to be marketed.

A non-limiting example of a hydrophilic proteins and polypeptides is human insulin which is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

WO2004147578 relates to fatty acid acylated amino acids used as permeation enhancers for hydrophobic molecules including hydrophobic macromolecules such as cyclosporine.

WO2001035998 relates to acylated amino acids used as transdermal and transmucosal absorption promoters for macromolecules, such as hydrophilic peptides or proteins.

WO2004064758 relates to an oral composition for delivering pharmaceutical peptides, such as insulin, growth hormone and GLP-1, comprising absorption enhancers, including acyl amino acids.

US2005282756 is related to a dry powder composition comprising insulin and an absorption enhancer.

WO2003030865 is related to insulin compositions comprising surfactants such as ionic surfactants and does also contain oil or lipid compounds such as triglycerides and does further comprise long chain esterified fatty acids (C12 to C18).

WO2004064758 is related to an oral pharmaceutical composition for delivering pharmaceutical peptides, comprising absorption enhancers.

The oral route of administration is rather complex and a need for establishment of an acceptable composition suitable for the treatment of patients, with an effective bioavailability of the macromolecule, such as hydrophilic peptides or proteins, is existent.

### SUMMARY

This invention is an oral pharmaceutical composition comprising certain amino acids acylated at their alpha-amino group with a fatty acid of 8 to 18 carbons and an active ingredient, such as a hydrophilic peptide or protein.

### BRIEF DESCRIPTION OF DRAWINGS

FIG. 1. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of a fatty acid acylated amino after injection into mid-jejunum of anaesthetized overnight fasted Sprague-Dawley rats (n=6)

FIG. 2. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of sodium N-capric leucine in two different concentrations after injection into mid-jejunum of anaesthetized overnight fasted Sprague-Dawley rats (n=4-6).

FIG. 3. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of two different fatty acid acylated amino acids after injection into mid-jejunum of anaesthetized overnight fasted Sprague-Dawley rats (n=6). The formulation (-□-) with N-cocoyl sarcosine contained 50% of the co-solvent propylene glycol. The fatty acid chain distribution in the cocoyl sarcosinate is 1% C6, 8% C8, 6% 010, 48% C12, 18% C14, 8% C16, 6% C18 saturated and 5% C18 unsaturated.

FIG. 4. Pharmakokinetic profiles of the insulin derivative A14E, B25H, B29K (N(eps)Octadecanedioyl-gGlu-OEG-OEG), desB30 human insulin (60 nmol/kg) dissolved in phosphate buffer (pH 7.4) in presence of increasing amounts of sodium lauroyl sarcosinate after injection into mid-jejunum of anaesthetized overnight fasted Sprague-Dawley rats (n=6).

FIG. 5. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of increasing amounts of sodium myristoyl glutamate after injection into mid-jejunum of anaesthetized overnight fasted Sprague-Dawley rats (n=4-6).

FIG. 6. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg/ml sodium lauroyl sarcosinate after injection into the colon of anaesthetized overnight fasted Sprague-Dawley rats.

FIG. 7. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of oleoyl sarcosinate or in presence of cocoyl sarcosinate and 16.5% of the co-solvent propylene glycol after injection into mid-jejunum of anaesthetized overnight fasted Sprague-Dawley rats (n=6). The fatty acid chain distribution in the cocoyl sarcosinate is 1% C6, 8% C8, 6% 010, 48% C12, 18% C14, 8% C16, 6% C18 saturated and 5% C18 unsaturated.

FIG. 8. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of different fatty acid acylated amino acids after injection into mid-jejunum of anaesthetized overnight fasted Sprague-Dawley rats (n=4-6).

FIG. 9. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of different fatty acid acylated amino acids after injection into mid-jejunum of anaesthetized overnight fasted Sprague-Dawley rats (n=5-6).

FIG. 10. Pharmakokinetic profiles of the insulin derivative A14E, B25H, B29K(N(eps)Octadecanedioyl-gGlu-OEG-OEG), desB30 human insulin (60 nmol/kg) dissolved in propylene glycol in presence of sodium N-capric leucine after injection into mid-jejunum of anaesthetized overnight fasted Sprague-Dawley rats (n=6).

FIG. 11. Pharmakokinetic profiles of the insulin derivative A14E, B25H, B29K(N(eps)Octadecanedioyl-gGlu-OEG-OEG), desB30 human insulin (120 nmol/kg) after peroral dosing of an enteric coated tablet comprising 200 mg of sodium lauroyl sarcosinate and 50 mg of soybean trypsine inhibitor and Eudragit® L30D55 and Eudragit® NE30D for enteric coating to male beagle dogs.

FIG. 12. Pharmakokinetic profiles of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of different fatty acid acylated amino acids or mixtures thereof according to the invention or in presence of commonly used permeation enhancers after injection into mid-jejunum of anaesthetized overnight fasted Sprague-Dawley rats (n=5-6).

FIG. 13. Pharmakokinetic profiles of the insulin derivative A14E, B25H, B29K(N(eps)Octadecanedioyl-gGlu-OEG-OEG), desB30 human insulin (30 nmol/kg) dissolved in liquid SEDDS, SMEDDS and SNEDDS formulations comprising sodium N-lauroyl phenylalanine after injection into mid-jejunum of anaesthetized overnight fasted Sprague-Dawley rats (n=5-6). The compositions are shown in table 1.

FIG. 14. Pharmakokinetic profile in a single beagle dog is shown of the insulin derivative A1 (N,N-Dimethyl), A14E, B1(N,N-dimethyl), B25H, B29K(N(eps)octadecanedioyl-gGlu-OEG-OEG), desB30 human insulin (120 nmol/kg) after peroral dosing of an enteric coated soft capsule comprising 30 mg of sodium lauroyl leucine sodium salt, 150 mg of propylene glycol, 300 mg of Polysorbate 20 and 520 mg of diglycerol monocaprylate. A 1:1 mixture of Euragit® L30-D55 and Eudragit® NE30D was used for enteric coating.

### DESCRIPTION

The present invention is related to pharmaceutical compositions, comprising FA-aa's acting as permeation enhancers suitable for oral administration of therapeutic macromolecules (e.i. therapeutic active peptides and proteins). More specifically therapeutic macromolecules, such as hydrophilic peptides or proteins according to the present invention are hydrophilic peptides and proteins which have a therapeutical activity and include but are not limited to insulin. The research into new surfactants with low irritant action has lead to the development of different surfactants derived from amino acids (Mitjans et al., 2003; Benavides et al., 2004; Sanchez et al., 2006) FA-aa's are amino acid based surfactants and thus mild biodegradable surfactants with a low toxicity.

It has surprisingly been found that certain fatty acid N-acylated amino acids increase the absorption of hydrophillic peptides and proteins after oral administration to a higher degree than commonly used permeation enhancers known in the art such as fatty acid salts, bile salts and others. This effect has been shown for hydrophilic peptides and proteins of varying sizes.

Due to their low toxicity and increasing effect on oral bioavailability of the therapeutic macromolecule, such as a hydrophilic peptide or protein, FA-aa's according to the present invention are valuable ingredients in oral pharmaceutical compositions. Especially valuable are FA-aa's according to this invention in oral pharmaceutical compositions comprising hydrophilic peptides or proteins as active ingredient. This is of interest for diseases that demand chronic administration of therapeutic macromolecules (e.g. peptides or proteins), but is not limited hereto, since the most non-invasive, non-toxic administration of drugs is generally favoured in any treatment, also for sporadic or bulk administration of therapeutics. So far, there are no commercial hydrophilic proteins available as oral formulation mainly due to the great challenges of enzymatic degradation and very low intestinal permeability of such hydrophilic proteins and peptides. Föger et al. described the impact of the molecular weight on oral absorption of hydrophilic peptide drugs and showed that the permeability decreased with increasing molecular weight of such hydrophilic peptide drugs (Amino Acids (2008) 25: 233-241, DOI 10.1007/s00726-007-0581-5).

The invention may also solve further problems that will be apparent from the disclosure of the exemplary embodiments. The present invention is related to oral pharmaceutical compositions comprising FA-aa's suitable for increasing the bioavailability of therapeutic macromolecules (e.g. peptides and proteins) and their absorption.

One embodiment of the invention is a pharmaceutical composition comprising at least one therapeutic macromolecule, such as hydrophilic peptides or proteins and at least one FA-aa. One embodiment of the invention is a pharmaceutical composition comprising at least one therapeutic macromolecule and at least one FA-aa, wherein said therapeutic macromolecule is ahydrophilic peptide or protein.

This invention also relates to a pharmaceutical composition comprising at least one therapeutic macromolecule and at least one FA-aa, wherein said therapeutic macromolecule is a peptide.

This invention also relates to a pharmaceutical composition comprising at least one therapeutic macromolecule and at least one FA-aa, wherein said therapeutic macromolecule is a therapeutic active peptide.

One embodiment of the invention is a pharmaceutical composition comprising at least one therapeutic macromolecule and at least one FA-aa, wherein said therapeutic macromolecule is a protein.

One embodiment of the invention is a pharmaceutical composition comprising at least one therapeutic macromolecule and at least one FA-aa, wherein said therapeutic macromolecule is a therapeutic protein.

One embodiment of the invention is a pharmaceutical composition comprising at least one therapeutic macromolecule and at least one FA-aa, wherein said therapeutic macromolecule is an insulin.

One embodiment of the invention is a pharmaceutical composition comprising at least one therapeutic macromolecule and at least one FA-aa, wherein said therapeutic macromolecule is an insulin peptide.

In one embodiment of the invention, the pharmaceutical composition comprises at least one therapeutic macromolecule and one or more FA-aa, based on a nonpolar hydrophobic amino acid.

In one embodiment of the invention, the pharmaceutical composition comprises at least one therapeutic macromolecule and one or more FA-aa, based on a nonpolar hydrophobic amino acid, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule and one or more FA-aa, based on a nonpolar hydrophobic amino acid and a fatty acid moiety consisting of 8 to 18 carbon atoms.

In one embodiment of the invention, the pharmaceutical composition comprises at least one therapeutic macromolecule and one or more FA-aa, based on a nonpolar hydrophobic amino acid and a fatty acid moiety consisting of 8 to 18 carbon atoms, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule and one or more FA-aa, based on a nonpolar hydrophobic amino acid and a fatty acid moiety consisting of 10 carbon atoms.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 10 carbon atoms, said one or more nonpolar hydrophobic amino acid may be selected from the group consisting Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 12 carbon atoms.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 12 carbon atoms, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 14 carbon atoms.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 14 carbon atoms, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 16 carbon atoms.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 16 carbon atoms, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 18 carbon atoms.

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 18 carbon atoms, said one or more nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment of the invention, the pharmaceutical composition comprises at least one therapeutic macromolecule, such as a hydrophilic peptide or protein and one or more FA-aa, based on a nonpolar hydrophobic amino acid, said nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, on a nonpolar hydrophobic amino acid and a fatty acid moiety consisting of 8 to 18 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 10 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 12 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 14 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 16 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a nonpolar hydrophobic amino acid and a fatty acid moiety consisting of 18 carbon atoms, said one or more nonpolar hydrophobic amino acid may be selected from the group consisting of Alanine (Ala), Valine (Val), Leucine (Leu), Phenylalanine (Phe).

In one embodiment of the invention, the pharmaceutical composition comprises at least one therapeutic macromolecule, such as a hydrophilic peptide or protein and one or more FA-aa, based on a polar uncharged amino acid.

In one embodiment of the invention, the pharmaceutical composition comprises at least one therapeutic macromolecule, such as a hydrophilic peptide or protein and one or more FA-aa, based on a polar uncharged amino acid, said polar uncharged amino acid may be selected from the group consisting of Glycine (Gly), Serine (Ser), Threonine (Thr), Cysteine (Cys), Tyrosine (Tyr), Asparagine (Apn), and Glutamine (Gln).

In one embodiment of the invention, the pharmaceutical composition comprises at least one therapeutic macromolecule, such as a hydrophilic peptide or protein and one or more FA-aa, based on a polar acidic amino acid.

In one embodiment of the invention, the pharmaceutical composition comprises at least one therapeutic macromolecule, such as a hydrophilic peptide or protein and one or more FA-aa, based on a polar acidic amino acid, said polar acidic amino acid may be selected from the group consisting of Aspartic acid (Asp) and Glutamic acid (Glu).

In one embodiment of the invention, the pharmaceutical composition comprises at least one therapeutic macromolecule, such as a hydrophilic peptide or protein and FA-aa's, based on a mixture FA-aa's.

In one embodiment a pharmaceutical composition according to the present invention comprises one or more commercially available FA-aa's.

According to this invention a FA-aa comprises an amino residue and a fatty acid attached to the amino acid by acylation of said amino acid's alpha-amino group.

In one embodiment, an amino acid residue according to this invention includes the form of its free acid or a salt.

In one embodiment an amino acid residue according to this invention includes the form of its free acid or sodium (Na+) salt.

In one embodiment a FA-aa according to this invention comprises an acylated amino acid and a fatty acid moiety consisting of 8 to 18 carbon atoms.

In one embodiment a FA-aa according to this invention comprises an acylated amino acid and a fatty acid moiety consisting of 10 carbon atoms.

In one embodiment a FA-aa according to this invention comprises an acylated amino acid and a fatty acid moiety consisting of 12 carbon atoms.

In one embodiment a FA-aa according to this invention comprises an acylated amino acid and a fatty acid moiety consisting of 14 carbon atoms.

In one embodiment a FA-aa according to this invention comprises an acylated amino acid and a fatty acid moiety consisting of 16 carbon atoms.

In one embodiment a FA-aa according to this invention comprises an acylated amino acid and a fatty acid moiety consisting of 18 carbon atoms.

In one embodiment a FA-aa comprises an amino acid residue acylated with a fatty acid or salt thereof.

In one embodiment a FA-aa according to this invention comprises an acylated amino acid, wherein the fatty acid moiety is located at the alpha amino group of the amino acid.

In one embodiment a FA-aa according to this invention comprises an acylated amino acid, wherein the fatty acid moiety is consisting of 8 to 18 carbon atoms.

In one embodiment a FA-aa according to this invention comprises an acylated amino acid, wherein the fatty acid moiety is consisting of 10 carbon atoms. In one embodiment a FA-aa according to this invention comprises an acylated amino acid, wherein the fatty acid moiety is consisting of 12 carbon atoms. In one embodiment a FA-aa according to this invention comprises an acylated amino acid, wherein the fatty acid moiety is consisting of 14 carbon atoms. In one embodiment a FA-aa according to this invention comprises an acylated amino acid, wherein the fatty acid moiety is consisting of 16 carbon atoms. In one embodiment a FA-aa according to this invention comprises an acylated amino acid, wherein the fatty acid moiety is consisting of 18 carbon atoms.

In one embodiment a FA-aa according to this invention comprises an acylated amino acid, wherein the fatty acid moiety is in the form of its free acid or salt. In one embodiment a FA-aa according to this invention comprises an acylated amino acid, wherein the fatty acid moiety is in the form of its free acid or sodium (Na+) salt. In one embodiment a FA-aa according to this invention comprise amino acid residues in the form of their free acid or a salt. In one embodiment a FA-aa according to this invention comprises amino acid residues in the form of their free acid or a sodium (Na+) salt. In one embodiment a FA-aa according to this invention is soluble at intestinal pH values, particularly in the 5.5 to 8.0 range. In one embodiment a FA-aa according to this invention is soluble at intestinal pH values, particularly in the 6.5 to 7.0 range.

In one embodiment a FA-aa according to this invention has a solubility of at least 5 mg/mL. In one embodiment a FA-aa according to this invention has a solubility of at least 10 mg/mL. In one embodiment a FA-aa according to this invention has a solubility of at least 20 mg/mL. In one embodiment a FA-aa according to this invention has a solubility of at least 30 mg/mL. In one embodiment a FA-aa according to this invention has a solubility of at least 40 mg/mL. In one embodiment a FA-aa according to this invention has a solubility of at least 50 mg/mL. In one embodiment a FA-aa according to this invention has a solubility of at least 60 mg/mL. In one embodiment a FA-aa according to this invention has a solubility of at least 70 mg/mL. In one embodiment a FA-aa according to this invention has a solubility of at least 80 mg/mL. In one embodiment a FA-aa according to this invention has a solubility of at least 90 mg/mL. In one embodiment a FA-aa according to this invention has a solubility of at least 100 mg/mL.

In one embodiment a FA-aa according to this invention has a solubility of at least 5 mg/mL in water. In one embodiment a FA-aa according to this invention has a solubility of at least 10 mg/mL in water. In one embodiment a FA-aa according to this invention has a solubility of at least 20 mg/mL in water. In one embodiment a FA-aa according to this invention has a solubility of at least 30 mg/mL in water. In one embodiment a FA-aa according to this invention has a solubility of at least 40 mg/mL in water. In one embodiment a FA-aa according to this invention has a solubility of at least 50 mg/mL in water. In one embodiment a FA-aa according to this invention has a solubility of at least 60 mg/mL in water. In one embodiment a FA-aa according to this invention has a solubility of at least 70 mg/mL in water. In one embodiment a FA-aa according to this invention has a solubility of at least 80 mg/mL in water. In one embodiment a FA-aa according to this invention has a solubility of at least 90 mg/mL in water. In one embodiment a FA-aa according to this invention has a solubility of at least 100 mg/mL in water.

A FA-aa according to the present invention may be represented by the general formula A-X, wherein A is an amino acid residue, based on a non-cationic amino acid and X is a fatty acid attached by acylation to A's alpha amino group.

In one embodiment a FA-aa according to the present invention may be represented by the general formula A-X, wherein A is an amino acid residue, based on a nonpolar hydrophobic amino acid and X is a fatty acid attached by acylation to A's alpha amino group.

In one embodiment a FA-aa according to the present invention may be represented by the general formula A-X, wherein A is an amino acid residue, based on a polar uncharges amino acid and X is a fatty acid attached by acylation to A's alpha amino group.

In one embodiment a FA-aa according to the present invention may be represented by the general formula A-X, wherein A is an amino acid residue, based on a polar acidic amino acid and X is a fatty acid attached by acylation to A's alpha amino group.

A FA-aa according to the present invention may be represented by the general formula;

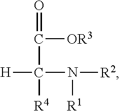

wherein R1 is a fatty acid chain comprising between from 8 to 18 carbons, R2 is either H (i.e. hydrogen) or CH₃ (i.e. methyl group), R3 is either H, or a salt thereof, and R4 is an amino acid side chain of a non-cationic amino acid.

In one embodiment a FA-aa according to the present invention may be represented by the general formula:

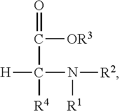

wherein R1 is a fatty acid chain comprising 8 to 18 carbon atoms, R2 is either H (i.e. hydrogen) or CH₃ (i.e. methyl group), R3 is either H, or a sodium salt (Na⁺) thereof, and R4 is a amino acid side chain of a non-cationic amino acid.

In one embodiment a FA-aa according to this invention may be chosen from the group consisting of formula (a), (b), (c), (d), (e), (f), (g), (h), (i), (j), (k), (l), (m), (n), (o), (p), (q) or (r), wherein R1 is a fatty acid chain comprising between from 8 to 18 carbons, R2 is either H (i.e. hydrogen) or CH₃ (i.e. methyl group), and R3 is either H, or a salt thereof.

In one embodiment a FA-aa according to this invention may be chosen from the group consisting of formula (a), (b), (c), (d), (e), (f), (g), (h), (i), (j), (k), (l), (m), (n), (o), (p), (q) or (r), wherein R1 is a fatty acid chain comprising 8 to 18 carbon atoms, R2 is either H (i.e. hydrogen) or CH₃ (i.e. methyl group), and R3 is either H, or a sodium (Na⁺) salt thereof.

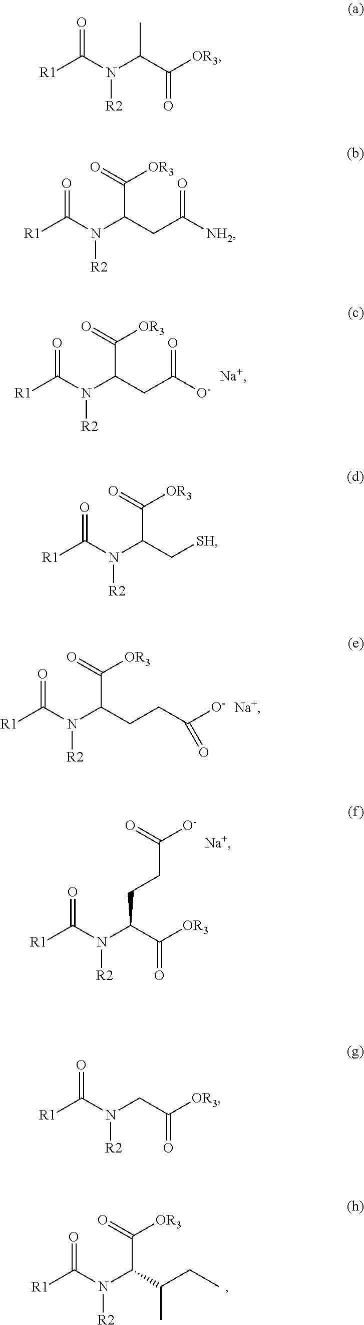

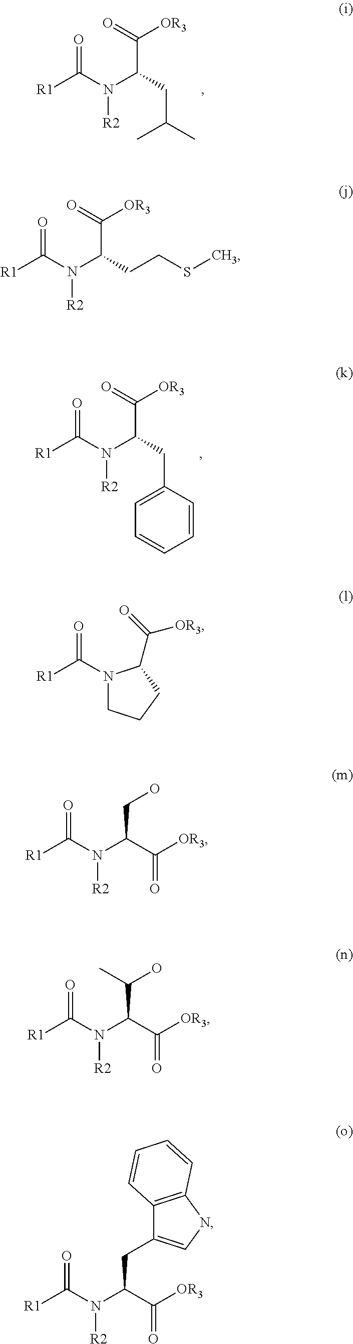

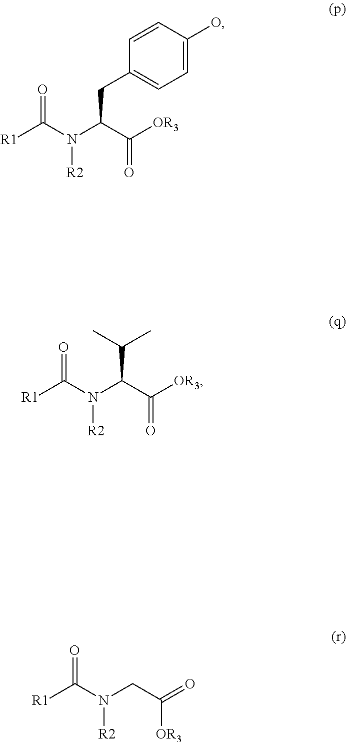

An amino acid residue according to this invention may be based on a non-cationic amino acid.

An amino acid residue according to this invention may be based on a non-cationic amino acid, said non-cationic amino acid may be selected from the group consisting nonpolar hydrophobic amino acids, polar uncharged amino acids and polar acidic amino acids.

An amino acid residue according to this invention may be based on a non-cationic amino acid, said non-cationic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).

In one embodiment an amino acid residue of a FA-aa according to this invention may be based on a nonpolar hydrophobic amino acid.

In one embodiment an amino acid residue of a FA-aa according to this invention may be based on a nonpolar hydrophobic amino acid, said nonpolar hydrophobic amino acid may be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment a FA-aa according to this invention comprises an acylated amino acid based on a nonpolar hydrophobic amino acid and a fatty acid moiety consisting of 8 to 18 carbon atoms, said nonpolar hydrophobic amino acid can be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment a FA-aa according to this invention comprises an acylated amino acid based on a nonpolar hydrophobic amino acid and a fatty acid moiety consisting of 10 carbon atoms.

In one embodiment a FA-aa according to this invention comprises an acylated amino acid based on a nonpolar hydrophobic amino acid and a fatty acid moiety consisting of 10 carbon atoms, said nonpolar hydrophobic amino acid can be selected from the group consisting Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment a FA-aa can be selected from the group consisting of: Sodium capric alaninate, N-decanoyl-L-alanine, Sodium capric isoleucinate, N-decanoyl-L-isoleucine, Sodium capric leucinate, N-decanoyl-L-leucine, Sodium capric methioninate, N-decanoyl-L-methionine, Sodium capric phenylalaninate, N-decanoyl-L-phenylalanine, Sodium capric prolinate, N-decanoyl-L-proline, Sodium capric threoninate, N-decanoyl-L-threonine, Sodium capric tryptophanate, N-decanoyl-L-tryptophane, Sodium capric valinate, N-decanoyl-L-valine, Sodium capric sarcosinate and N-decanoyl-L-sarcosine.

In one embodiment a FA-aa according to this invention comprises an acylated amino acid based on a nonpolar hydrophobic amino acid and a fatty acid moiety consisting of 12 carbon atoms.

In one embodiment a FA-aa according to this invention comprises an acylated amino acid based on a nonpolar hydrophobic amino acid and a fatty acid moiety consisting of 12 carbon atoms, said nonpolar hydrophobic amino acid can be selected from the group consisting of Alanine (Ala), Valine (Val), Leucine (Leu), Isoleucine (Ile), Phenylalanine (Phe), Tryptophane (Trp), Metheonine (Met), Proline (Pro) and Sarcosinate.

In one embodiment a FA-aa can be selected from the group consisting of: Sodium lauroyl alaninate, N-dodecanoyl-L-alanine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl tryptophanate, N-dodecanoyl-L-tryptophane, Sodium lauroyl valinate, N-dodecanoyl-L-valine, Sodium lauroyl sarcosinate, N-dodecanoyl-L-sarcosine, Sodium lauroyl sarcosinate, Sodium oleoyl sarcosinate and Sodium N-decyl leucine.

In one embodiment a FA-aa according to this invention comprises an acylated amino acid based on a nonpolar hydrophobic amino acid, said nonpolar hydrophobic amino acid can be selected from the group consisting of Alanine (Ala), Valine (Val), Leucine (Leu), Phenylalanine (Phe).

In one embodiment a FA-aa according to this invention comprises an acylated amino acid based on a nonpolar hydrophobic amino acid and a fatty acid moiety consisting of 8 to 18 carbon atoms, said nonpolar hydrophobic amino acid can be selected from the group consisting of Alanine (Ala), Valine (Val), Leucine (Leu), Phenylalanine (Phe).

In one embodiment a FA-aa according to this invention comprises an acylated amino acid based on a nonpolar hydrophobic amino acid and a fatty acid moiety consisting of 10 carbon atoms. In one embodiment a FA-aa according to this invention comprises an acylated amino acid based on a nonpolar hydrophobic amino acid and a fatty acid moiety consisting of 10 carbon atoms, said nonpolar hydrophobic amino acid can be selected from the group consisting of Alanine (Ala), Valine (Val), Leucine (Leu), Phenylalanine (Phe).

In one embodiment a FA-aa can be selected from the group consisting of: Sodium capric alaninate, N-decanoyl-L-alanine, Sodium capric leucinate, N-decanoyl-L-leucine, Sodium capric phenylalaninate, N-decanoyl-L-phenylalanine, Sodium capric valinate, N-decanoyl-L-valine, Sodium N-decyl leucine,

In one embodiment a FA-aa according to this invention comprises an acylated amino acid based on a nonpolar hydrophobic amino acid and a fatty acid moiety consisting of 12 carbon atoms. In one embodiment a FA-aa according to this invention comprises an acylated amino acid based on a nonpolar hydrophobic amino acid and a fatty acid moiety consisting of 12 carbon atoms, said nonpolar hydrophobic amino acid can be selected from the group consisting of Alanine (Ala), Valine (Val), Leucine (Leu), Phenylalanine (Phe).

In one embodiment a FA-aa according to this invention comprises an acylated amino acid based on a nonpolar hydrophobic amino acid and a fatty acid moiety consisting of 14 carbon atoms. In one embodiment a FA-aa according to this invention comprises an acylated amino acid based on a nonpolar hydrophobic amino acid and a fatty acid moiety consisting of 14 carbon atoms, said nonpolar hydrophobic amino acid can be selected from the group consisting of Alanine (Ala), Valine (Val), Leucine (Leu), Phenylalanine (Phe).

In one embodiment a FA-aa according to this invention comprises an acylated amino acid based on a nonpolar hydrophobic amino acid and a fatty acid moiety consisting of 16 carbon atoms. In one embodiment a FA-aa according to this invention comprises an acylated amino acid based on a nonpolar hydrophobic amino acid and a fatty acid moiety consisting of 16 carbon atoms, said nonpolar hydrophobic amino acid can be selected from the group consisting of Alanine (Ala), Valine (Val), Leucine (Leu), Phenylalanine (Phe).

In one embodiment a FA-aa according to this invention comprises an acylated amino acid based on a nonpolar hydrophobic amino acid and a fatty acid moiety consisting of 18 carbon atoms. In one embodiment a FA-aa according to this invention comprises an acylated amino acid based on a nonpolar hydrophobic amino acid and a fatty acid moiety consisting of 18 carbon atoms, said nonpolar hydrophobic amino acid can be selected from the group consisting of Alanine (Ala), Valine (Val), Leucine (Leu), Phenylalanine (Phe).

In one embodiment a FA-aa can be selected from the group consisting of: Sodium lauroyl alaninate, N-dodecanoyl-L-alanine, Sodium lauroyl leucinate, N-dodecanoyl-L-leucine, Sodium lauroyl phenylalaninate, N-dodecanoyl-L-phenylalanine, Sodium lauroyl valinate, N-dodecanoyl-L-valine, In one embodiment an amino acid residue of a FA-aa according to this invention can be based on a polar uncharged amino acid.

In one embodiment an amino acid residue of a FA-aa according to this invention can be based on a polar uncharged amino acid, said polar uncharged amino acid can be selected from the group consisting of Glycine (Gly), Serine (Ser), Threonine (Thr), Cysteine (Cys), Tyrosine (Tyr), Asparagine (Apn), and Glutamine (Gln).

In one embodiment a FA-aa can be selected from the group consisting of: Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutaminate, N-dodecanoyl-L-glutamine, Sodium lauroyl glycinate, N-dodecanoyl-L-glycine, Sodium lauroyl serinate, N-dodecanoyl-L-serine, Sodium lauroyl threoninate, N-dodecanoyl-L-threonine, Sodium lauroyl tyrosinate, N-dodecanoyl-L-tyrosine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutaminate, N-decanoyl-L-glutamine, Sodium capric glycinate, N-decanoyl-L-glycine, Sodium capric serinate, N-decanoyl-L-serine, Sodium capric threoninate, N-decanoyl-L-threonine, Sodium capric tyrosinate and N-decanoyl-L-tyrosine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutaminate, N-dodecanoyl-L-glutamine, Sodium lauroyl glycinate, N-dodecanoyl-L-glycine, Sodium lauroyl serinate, N-dodecanoyl-L-serine, Sodium lauroyl threoninate, N-dodecanoyl-L-threonine, Sodium lauroyl tyrosinate, N-dodecanoyl-L-tyrosine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutaminate, N-decanoyl-L-glutamine, Sodium capric glycinate, N-decanoyl-L-glycine, Sodium capric serinate, N-decanoyl-L-serine, Sodium capric threoninate, N-decanoyl-L-threonine, Sodium capric tyrosinate and N-decanoyl-L-tyrosine.

In one embodiment an amino acid residue of a FA-aa according to this invention can be based on a polar acidic amino acid.

In one embodiment an amino acid residue of a FA-aa according to this invention can be based on a polar acidic amino acid, said polar acidic amino acid can be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 10 carbon atoms, said nonpolar hydrophobic amino acid can be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 12 carbon atoms, said nonpolar hydrophobic amino acid can be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 14 carbon atoms, said nonpolar hydrophobic amino acid can be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 16 carbon atoms, said nonpolar hydrophobic amino acid can be selected from the group consisting of Aspartic acid (Asp) and Glutamic acid (Glu).

In one embodiment a FA-aa can be selected from the group consisting of: Sodium lauroyl asparaginate, N-dodecanoyl-L-asparagine, Sodium lauroyl aspartic acid, N-dodecanoyl-L-aspartic acid, Sodium lauroyl glutamic acid, N-dodecanoyl-L-glutamic acid, Sodium capric asparaginate, N-decanoyl-L-asparagine, Sodium capric aspartic acid, N-decanoyl-L-aspartic acid, Sodium capric glutamic acid and N-decanoyl-L-glutamic acid.

In one embodiment a FA-aa can be selected from the group consisting of: Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate) and Sodium cocoyl glutamate, Sodium lauroyl asparaginate, N-dodecanoyl-L-asparagine, Sodium lauroyl aspartic acid, N-dodecanoyl-L-aspartic acid, Sodium lauroyl glutamic acid, N-dodecanoyl-L-glutamic acid, Sodium capric asparaginate, N-decanoyl-L-asparagine, Sodium capric aspartic acid, N-decanoyl-L-aspartic acid, Sodium capric glutamic acid and N-decanoyl-L-glutamic acid.

In one embodiment a FA-aa can be selected from the group consisting of: Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate) and Sodium cocoyl glutamate.

In one embodiment an amino acid residue of a FA-aa according to this invention may be based on a polar acidic amino acid.

In one embodiment an amino acid residue of a FA-aa according to this invention may be based on a polar acidic amino acid, said polar acidic amino acid may be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 14 carbon atoms, said nonpolar hydrophobic amino acid may be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 16 carbon atoms, said nonpolar hydrophobic amino acid may be selected from the group consisting of Aspartic acid (Asp) and Glutamic acid (Glu).

In one embodiment a FA-aa according to this invention comprises an acylated amino acid based on a polar acidic amino acid and a fatty acid moiety consisting of 18 carbon atoms, said nonpolar hydrophobic amino acid can be selected from the group consisting of Aspartic acid (Asp) and Glutamic acid (Glu).

In one embodiment a FA-aa may be selected from the group consisting of: Sodium lauroyl asparaginate, N-dodecanoyl-L-asparagine, Sodium lauroyl aspartic acid, N-dodecanoyl-L-aspartic acid, Sodium lauroyl glutamic acid, N-dodecanoyl-L-glutamic acid, Sodium capric asparaginate, N-decanoyl-L-asparagine, Sodium capric aspartic acid, N-decanoyl-L-aspartic acid, Sodium capric glutamic acid and N-decanoyl-L-glutamic acid.

In accordance with the present invention an amino acid amino acid may be selected from the group consisting of Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate) and Sodium cocoyl glutamate.

In accordance with the present invention an amino acid amino acid FA-aa may be selected from the group consisting of: Sodium lauroyl asparaginate, N-dodecanoyl-L-asparagine, Sodium lauroyl aspartic acid, N-dodecanoyl-L-aspartic acid, Sodium lauroyl glutamic acid, N-dodecanoyl-L-glutamic acid, Sodium capric asparaginate, N-decanoyl-L-asparagine, Sodium capric aspartic acid, N-decanoyl-L-aspartic acid, Sodium capric glutamic acid and N-decanoyl-L-glutamic acid.

In accordance with the present invention an amino acid amino acid may be selected from the group consisting of Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate) and Sodium cocoyl glutamate.

In one embodiment an amino acid moiety of a FA-aa according to this invention is an amino acids that are not encoded by the genetic code.

In one embodiment an amino acid moiety of a FA-aa according to this invention is Sarcosinate.

In one embodiment an amino acid residue of a FA-aa according to this invention is an free acid or salt form of an amino acid that are not encoded by the genetic code.

In one embodiment an amino acid residue of a FA-aa according to this invention is the free acid or salt form of Sarcosinate.

In one embodiment an amino acid moiety of a FA-aa according to this invention is selected from the group comprising Leucine and Phenylalanine.

Modifications of amino acids by acylation are readily performed using acylation agents known in the art that react with the free alpha-amino group of the amino acid.

The following FA-aa's are commercially available:

| Brand name | Chemical name | Provider (2011-04-14) |
|---|---|---|
| Hamposyl L-95 | Sodium lauroyl sarcosine | Chattem Chemicals |
| Hamposyl O | Sodium oleoyl sarcosine | Chattem Chemicals |
| Hamposyl C | Sodium cocoyl sarcosine | Chattem Chemicals |
| Hamposyl L-30 | Sodium lauroyl sarcosine | Chattem Chemicals |
| Amisoft HS-11 P | Sodium stearoyl glutamate | Ajinomoto |
| Amisoft LS-11 | Sodium lauroyl glutamate | Ajinomoto |
| Amisoft CS-11 | Sodium cocoyl glutamate | Ajinomoto |
| Amisoft MS-11 | Sodium myristoyl glutamate | Ajinomoto |
| Amilite GCS-11 | Sodium cocoyl glycinate | Ajinomoto |

According to the present invention, the FA-aa may be part of an oral pharmaceutical composition.

In one embodiment of the invention the pharmaceutical composition comprises of at least one therapeutic macromolecule, such as a hydrophilic peptide or protein and at least on FA-aa and propylene glycol.

In one embodiment the amino acid FA-aa may be used in a liquid or semisolid liquid and surfactant based delivery system. In one embodiment the amino acid FA-aa may be used in a liquid or semisolid liquid and surfactant based delivery system, such as SEDDS, SMEDDS or SNEDDS. In one embodiment the amino acid FA-aa may be used in a solid surfactant based delivery system. In one embodiment the amino acid FA-aa may be used in a solid surfactant based delivery system, such as SEDDS, SMEDDS or SNEDDS. Liquid or semisolid SEDDS, SMEDDS or SNEDDS comprising FA-aa's according to the invention may be encapsulated with any available soft- or hard capsule technology to result in a solid oral pharmaceutical dosage form. Thus the term “solid” as used herein refers to liquid compositions encapsulated in a soft or hard capsule technology, but also to tablets and multiparticulates.

Liquid or semisolid SEDDS, SMEDDS or SNEDDS according to the invention may be encapsulated with any available soft- or hard capsule technology to result in a solid oral pharmaceutical dosage form which may further comprise enteric or delayed release coatings.

Liquid or semisolid SEDDS, SMEDDS or SNEDDS comprising FA-aa's according to the invention may be encapsulated with any available soft- or hard capsule technology to result in a solid oral pharmaceutical dosage form which may further comprise enteric or delayed release coatings, such as poly(meth)acrylates, commercially known as Eudragit®.

In one embodiment of the invention the pharmaceutical composition is a SEDDS, SMEDDS or SNEDDS, comprising at least one therapeutic macromolecule, such as a hydrophilic peptide or protein and at least one FA-aa, propylene glycol.

In one embodiment the pharmaceutical composition according to the present comprises less than 10% (w/w) water. In one embodiment the pharmaceutical composition according to the present comprises less than 9% (w/w) water. In one embodiment the pharmaceutical composition according to the present comprises less than 8% (w/w) water. In one embodiment the pharmaceutical composition according to the present comprises less than 7% (w/w) water. In one embodiment the pharmaceutical composition according to the present comprises less than 6% (w/w) water. In one embodiment the pharmaceutical composition according to the present comprises less than 5% (w/w) water. In one embodiment the pharmaceutical composition according to the present comprises less than 4% (w/w) water. In one embodiment the pharmaceutical composition according to the present comprises less than 3% (w/w) water. In one embodiment the pharmaceutical composition according to the present comprises less than 2% (w/w) water. In one embodiment the pharmaceutical composition according to the present comprises less than 1% (w/w) water. In one embodiment the pharmaceutical composition according to the present comprises less than 0% (w/w) water.

In one embodiment the pharmaceutical composition according to the present invention is a liquid. In one embodiment the pharmaceutical composition according to the present invention is a liquid and comprises less than 10% (w/w) water. In one embodiment the pharmaceutical composition according to the present invention is a liquid and comprises less than 9% (w/w) water. In one embodiment the pharmaceutical composition according to the present invention is a liquid and comprises less than 8% (w/w) water. In one embodiment the pharmaceutical composition according to the present invention is a liquid and comprises less than 7% (w/w) water. In one embodiment the pharmaceutical composition according to the present invention is a liquid and comprises less than 6% (w/w) water. In one embodiment the pharmaceutical composition according to the present invention is a liquid and comprises less than 5% (w/w) water. In one embodiment the pharmaceutical composition according to the present invention is a liquid and comprises less than 4% (w/w) water. In one embodiment the pharmaceutical composition according to the present invention is a liquid and comprises less than 3% (w/w) water. In one embodiment the pharmaceutical composition according to the present invention is a liquid and comprises less than 2% (w/w) water. In one embodiment the pharmaceutical composition according to the present invention is a liquid and comprises less than 1% (w/w) water. In one embodiment the pharmaceutical composition according to the present invention is a liquid and comprises less than 0% (w/w) water.

In one embodiment of the invention the pharmaceutical composition comprises at least one therapeutic macromolecule. In one embodiment a therapeutic macromolecule, such as a hydrophilic peptide or protein according to this invention is a therapeutic active peptide or protein. In one embodiment a therapeutic peptide or protein according to this invention is a hydrophilic peptide or protein.

In one embodiment a hydrophilic peptide or protein of this invention is a peptide or protein having a solubility of at least 50 mg/mL in water. In one embodiment a hydrophilic peptide or protein of this invention is a peptide or protein having a solubility of at least 60 mg/mL in water In one embodiment a hydrophilic peptide or protein of this invention is a peptide or protein having a solubility of at least 70 mg/mL in water In one embodiment a hydrophilic peptide or protein of this invention is a peptide or protein having a solubility of at least 80 mg/mL in water. In one embodiment a hydrophilic peptide or protein of this invention is a peptide or protein having a solubility of at least 90 mg/mL in water. In one embodiment a hydrophilic peptide or protein of this invention is a peptide or protein having a solubility of at least 100 mg/mL in water. In one embodiment a hydrophilic peptide or protein of this invention is a peptide or protein having a solubility of at least 110 mg/mL in water. In one embodiment a hydrophilic peptide or protein of this invention is a peptide or protein having a solubility of at least 120 mg/mL in water. In one embodiment a hydrophilic peptide or protein of this invention is a peptide or protein having a solubility of at least 130 mg/mL in water. In one embodiment a hydrophilic peptide or protein of this invention is a peptide or protein having a solubility of at least 140 mg/mL in water. In one embodiment a hydrophilic peptide or protein of this invention is a peptide or protein having a solubility of at least 150/mL in water. In one embodiment a hydrophilic peptide or protein of this invention is a peptide or protein having a solubility of at least 160 mg/mL in water. In one embodiment a hydrophilic peptide or protein of this invention is a peptide or protein having a solubility of at least 170 mg/mL in water. In one embodiment a hydrophilic peptide or protein of this invention is a peptide or protein having a solubility of at least 180 mg/mL in water. In one embodiment a hydrophilic peptide or protein of this invention is a peptide or protein having a solubility of at least 190 mg/mL in water. In one embodiment a hydrophilic peptide or protein of this invention is a peptide or protein having a solubility of at least 200 mg/mL in water. In one embodiment a hydrophilic peptide or protein of this invention is a peptide or protein having a solubility of at least 210 mg/mL in water. In one embodiment a hydrophilic peptide or protein of this invention is a peptide or protein having a solubility of at least 220 mg/mL in water. In one embodiment a hydrophilic peptide or protein of this invention is a peptide or protein having a solubility of at least 230 mg/mL in water. In one embodiment a hydrophilic peptide or protein of this invention is a peptide or protein having a solubility of at least 240 mg/mL in water.

In one embodiment a therapeutic active peptide or protein according to this invention is a peptide or protein of more than 1500 Da. In one embodiment a therapeutic active peptide or protein according to this invention is a peptide or protein of more than 1750 Da. In one embodiment a therapeutic active peptide or protein according to this invention is a peptide or protein of more than 2000 Da. In one embodiment a therapeutic active peptide or protein according to this invention is a peptide or protein of more than 2250 Da. In one embodiment a therapeutic active peptide or protein according to this invention is a peptide or protein of more than 2500 Da. In one embodiment a therapeutic active peptide or protein according to this invention is a peptide or protein of more than 2750 Da. In one embodiment a therapeutic active peptide or protein according to this invention is a peptide or protein of more than 3000 Da. In one embodiment a therapeutic active peptide or protein according to this invention is a peptide or protein of more than 3250 Da. In one embodiment a therapeutic active peptide or protein according to this invention is a peptide or protein of more than 3500 Da. In one embodiment a therapeutic active peptide or protein according to this invention is a peptide or protein of more than 3750 Da. In one embodiment a therapeutic active peptide or protein according to this invention is a peptide or protein of more than 4000 Da. In one embodiment a therapeutic active peptide or protein according to this invention is a peptide or protein of more than 4250 Da. In one embodiment a therapeutic active peptide or protein according to this invention is a peptide or protein of more than 4500 Da. In one embodiment a therapeutic active peptide or protein according to this invention is a peptide or protein of more than 4750 Da. In one embodiment a therapeutic active peptide or protein according to this invention is a peptide or protein of more than 5000 Da. In one embodiment a therapeutic active peptide or protein according to this invention is a peptide or protein of more than 1500 Da. In one embodiment a therapeutic active peptide or protein according to this invention is a peptide or protein of between 1500 Da and 5000 Da.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent.

In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the solvent is selected from the group consisting of water and propylene glycol.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, wherein said Polyethylene glycol sorbitan fatty acid ester is selected from the group consisting of Tween 20, Tween 21, Tween 40, Tween 60, Tween 65, Tween 80, Tween 81 and Tween 85. In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester, wherein said Polyethylene glycol sorbitan fatty acid ester is selected from the group consisting of Tween 20, Tween 21, Tween 40, Tween 60, Tween 65, Tween 80, Tween 81 and Tween 85.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the solvent is selected from the group consisting of water and propylene glycol.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 85.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 85 and the solvent is selected form the group consisting of water and propylene glycol.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan trioleate commercially known as Tween 85 and a polar or semipolar solvent selected from the group consisting of water and propylene glycol, wherein the composition forms a microemulsion after dilution in an aqueous medium.

In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan trioleate commercially known as Tween 85 and a polar or semipolar solvent selected from the group consisting of water and propylene glycol, wherein the composition forms a microemulsion after dilution in an aqueous medium.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 20.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan monolaurate, commercially known as Tween 20 and the solvent is selected form the group consisting of water and propylene glycol.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan monolaurate commercially known as Tween 20 and a polar or semipolar solvent selected from the group consisting of water and propylene glycol, wherein the composition forms a microemulsion after dilution in an aqueous medium.

In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan monolaurate commercially known as Tween 20 and a polar or semipolar solvent selected from the group consisting of water and propylene glycol, wherein the composition forms a microemulsion after dilution in an aqueous medium.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent. In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent, wherein said polar or semipolar solvent is selected from the group consisting of water and propylene glycol. In one embodiment a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent, wherein said polar or semipolar solvent is selected from the group consisting of water and propylene glycol.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent (such as water or propylene glycol). In one embodiment a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester (Span 10, 20, 40, 60 or 80), and a polar or semipolar solvent (such as water or propylene glycol).

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester, wherein said sorbitan fatty acid ester is selected form the group consisting of Span 10, Span 20, Span 40, Span 60 and Span 80. In one embodiment a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester, wherein said sorbitan fatty acid ester is selected form the group consisting of sorbitan laurate commercially known as Span 20, sorbitan mono palmitate commercially known as Span 40, sorbitan mono stearate commercially known as Span 60 and sorbitan oleate commercially known as Span 80.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent. In one embodiment a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester and a polar or semipolar solvent.

In one embodiment a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent, wherein said polar or semipolar solvent is selected from the group consisting of water or propylene glycol.

In one embodiment of the invention the pharmaceutical composition comprises at least one therapeutic active peptide or protein.

In one embodiment of the present invention the pharmaceutical composition comprises at least one therapeutic active peptide or protein, which has been pH neutralised.

In one embodiment of the invention the therapeutical active peptide or protein is dissolved and the pH of the resulting solution is adjusted to a value of the target pH value, which is 1 unit, alternatively 2 units and alternatively 2.5 pH units above or below the pI of the insulin peptide, whereafter said resulting solution is freeze or spray dryed. In one embodiment said pH adjustment is performed with a non-volatile acid or base.

In one embodiment of the invention the pharmaceutical composition comprises of at least one insulin peptide and at least on FA-aa. In one embodiment of the invention the pharmaceutical composition comprises of at least one peptide or protein and at least on FA-aa.

In one embodiment of the invention the pharmaceutical composition comprises of at least one insulin peptide and at least on FA-aa and propylene glycol.

In one embodiment the amino acid FA-aa may be used in a liquid or semisolid liquid and surfactant based delivery system. In one embodiment the amino acid FA-aa may be used in a liquid or semisolid liquid and surfactant based delivery system comprising less than 10% (w/w) water. In one embodiment the amino acid FA-aa may be used in a liquid or semisolid liquid and surfactant based delivery system comprising less than 9% (w/w) water.

In one embodiment the amino acid FA-aa may be used in a liquid or semisolid liquid and surfactant based delivery system comprising less than 8% (w/w) water. In one embodiment the amino acid FA-aa may be used in a liquid or semisolid liquid and surfactant based delivery system comprising less than 7% (w/w) water. In one embodiment the amino acid FA-aa may be used in a liquid or semisolid liquid and surfactant based delivery system comprising less than 6% (w/w) water. In one embodiment the amino acid FA-aa may be used in a liquid or semisolid liquid and surfactant based delivery system comprising less than 5% (w/w) water. In one embodiment the amino acid FA-aa may be used in a liquid or semisolid liquid and surfactant based delivery system comprising less than 4% (w/w) water. In one embodiment the amino acid FA-aa may be used in a liquid or semisolid liquid and surfactant based delivery system comprising less than 3% (w/w) water. In one embodiment the amino acid FA-aa may be used in a liquid or semisolid liquid and surfactant based delivery system comprising less than 2% (w/w) water. In one embodiment the amino acid FA-aa may be used in a liquid or semisolid liquid and surfactant based delivery system comprising less than 1% (w/w) water. In one embodiment the amino acid FA-aa may be used in a liquid or semisolid liquid and surfactant based delivery system comprising less than 0% (w/w) water.

In one embodiment a pharmaceutical composition according to the present invention comprises at least one therapeutic hydrophilic protein or polypeptide, at least one fatty acid acylated amino acid, at least one high HLB surfactant, at least one low HLB co-surfactant and a polar solvent. In one embodiment a pharmaceutical composition according to the present invention comprises a therapeutic hydrophilic protein or polypeptide, at least one fatty acid acylated amino acid, at least one high HLB surfactant, at least one low HLB co-surfactant and a polar solvent.

In one embodiment a pharmaceutical composition according to the present invention comprises at least one therapeutic hydrophilic protein or polypeptide, at least one fatty acid acylated amino acid, at least two high HLB surfactants, and a polar solvent. In one embodiment a pharmaceutical composition according to the present invention comprises a therapeutic hydrophilic protein or polypeptide, at least one fatty acid acylated amino acid, at least two high HLB surfactants, and a polar solvent.

In one embodiment the amino acid FA-aa may be used in a liquid or semisolid liquid and surfactant based delivery system, such as SEDDS, SMEDDS or SNEDDS. In one embodiment the amino acid FA-aa may be used in a solid surfactant based delivery system comprising less than 10% (w/w) water. In one embodiment the amino acid FA-aa may be used in a solid surfactant based delivery system comprising less than 9% (w/w) water. In one embodiment the amino acid FA-aa may be used in a solid surfactant based delivery system comprising less than 8% (w/w) water. In one embodiment the amino acid FA-aa may be used in a solid surfactant based delivery system comprising less than 7% (w/w) water. In one embodiment the amino acid FA-aa may be used in a solid surfactant based delivery system comprising less than 6% (w/w) water. In one embodiment the amino acid FA-aa may be used in a solid surfactant based delivery system comprising less than 6% (w/w) water. In one embodiment the amino acid FA-aa may be used in a solid surfactant based delivery system comprising less than 5% (w/w) water. In one embodiment the amino acid FA-aa may be used in a solid surfactant based delivery system comprising less than 4% (w/w) water. In one embodiment the amino acid FA-aa may be used in a solid surfactant based delivery system comprising less than 3% (w/w) water. In one embodiment the amino acid FA-aa may be used in a solid surfactant based delivery system comprising less than 2% (w/w) water. In one embodiment the amino acid FA-aa may be used in a solid surfactant based delivery system comprising less than 1% (w/w) water. In one embodiment the amino acid FA-aa may be used in a solid surfactant based delivery system comprising less than 0% (w/w) water.

In one embodiment the amino acid FA-aa may be used in a solid surfactant based delivery system, such as SEDDS, SMEDDS or SNEDDS.

In one embodiment the pharmaceutical composition according to the present invention is a liquid.

In one embodiment pharmaceutical composition is a liquid or semisolid SEDDS, SMEDDS or SNEDDS comprising FA-aa's according to the invention and is encapsulated with any available soft- or hard capsule technology to result in a solid oral pharmaceutical dosage form. In one embodiment a soft capsule technology used for encapsulating a composition according to the present invention is gelatine free. In one embodiment a gelatine free soft capsule technology as commercially known under the name Vegicaps® from Catalent® is used for encapsulation of the pharmaceutical composition according to the present invention.

In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 10% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 9% (w/w) water In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 8% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 7% (w/w) water In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 6% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 5% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 4% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 3% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 2% (w/w) water. In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 1% (w/w) water In one embodiment the pharmaceutical composition a liquid or semisolid SEDDS, SMEDDS or SNEDDS comprising FA-aa's according to the invention and is encapsulated with any available soft- or hard capsule technology to result in a solid oral pharmaceutical dosage form comprising less than 0% (w/w) water.

In one embodiment a liquid or semisolid formulation according to the invention is encapsulated with any available soft- or hard capsule technology to result in a solid oral pharmaceutical dosage form further comprising an enteric or delayed release coatings.

In one embodiment a liquid or semisolid formulation according to the invention is encapsulated with any available enteric soft- or hard capsule technology to result in a solid oral pharmaceutical dosage.

In one embodiment a liquid or semisolid SEDDS, SMEDDS or SNEDDS comprising FA-aa's according to the invention is encapsulated with any available soft- or hard capsule technology to result in a solid oral pharmaceutical dosage form further comprising an enteric or delayed release coatings. In one embodiment a liquid or semisolid SEDDS, SMEDDS or SNEDDS comprising FA-aa's according to the invention is encapsulated with any available enteric soft- or hard capsule technology to result in a solid oral pharmaceutical dosage.

In one embodiment a liquid or semisolid SEDDS, SMEDDS or SNEDDS comprising FA-aa's according to the invention is encapsulated with any available soft- or hard capsule technology to result in a solid oral pharmaceutical dosage form which may further comprise an enteric or delayed release coatings, such as poly(meth)acrylates, commercially known as Eudragit®.

In one embodiment, the coating comprises at least one release modifying polymer which can be used to control the site where the drug (insulin derivative) is released. The modified release polymer can be a polymethacrylate polymer such as those sold under the Eudragit® trade name (Evonik Rohm GmbH, Darmstadt, Germany), for example Eudragit® L30 D55, Eudragit® L100-55, Eudragit® L100, Eudragit® S100, Eudragit® S12,5, Eudragit® FS30D, Eudragit® NE30D and mixtures thereof as e.g. described in Eudragit® Application Guidelines, Evonik Industries, 11th edition, 09/2009.

In one embodiment of the invention the pharmaceutical composition is a formulation, comprising at least one insulin and at least one FA-aa, propylene glycol.

In one embodiment of the invention the pharmaceutical composition comprises of at least one insulin and at least one FA-aa, propylene glycol.

In one embodiment of the invention the pharmaceutical comprises at least one peptide or protein and at least one FA-aa, propylene glycol.

In one embodiment of the invention the pharmaceutical composition is a SEDDS, SMEDDS or SNEDDS, comprising at least one peptide or protein and at least one FA-aa, propylene glycol.

The components of the drug delivery system may be present in any relative amounts. In one embodiment the drug delivery system comprises up to 90% of a surfactant, or up to 90% of a polar organic solvent such as Polyethylene glycol (PEG) 300 g/mol, PEG 400 g/mol, PEG 600 g/mol, PEG 1000 g/mol, or up to 90% of a lipid component. PEGs are prepared by polymerization of ethylene oxide and are commercially available over a wide range of molecular weights from 300 g/mol to 10,000,000 g/mol.

In one embodiment the oral pharmaceutical composition comprises from 5 to 20% of propylene glycol.

In one embodiment, the oral pharmaceutical composition comprises at least one FA-aa, propylene glycol, and at least two non ionic surfactants.

In one embodiment, the oral pharmaceutical composition comprises at least one FA-aa, propylene glycol, polysorbate 20 and a co-surfactant. Polysorbate 20 is a polysorbate surfactant whose stability and relative non-toxicity allows it to be used as a detergent and emulsifier in a number of domestic, scientific, and pharmacological applications. The number 20 refers to the total number of oxyethylene —(CH₂CH₂O)— groups found in the molecule.

In one embodiment of the present invention, the oral pharmaceutical composition comprises at least one FA-aa, propylene glycol, polysorbate 20 and a polyglycerol fatty acid ester.

In one embodiment, the oral pharmaceutical composition comprises at least one FA-aa, propylene glycol, polysorbate 20 and a co-surfactant.

In one embodiment, the oral pharmaceutical composition comprises at least one FA-aa, propylene glycol, polysorbate 20 and a polyglycerol fatty acid ester such as diglycerol monocaprylate.

In certain embodiments of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavors, sweeteners and other components as described in Handbook of Pharmaceutical Excipients, Rowe et al., Eds., 4th Edition, Pharmaceutical Press (2003), which is hereby incorporated by reference

These additional excipients may be in an amount from about 0.05-5% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavouring agents typically provide up to about 2.5% or 5% by weight of the total pharmaceutical composition.

Oral pharmaceutical compositions according to this invention may be formulated as solid dosage forms.

Oral pharmaceutical compositions according to this invention may be formulated as solid dosage forms and may be selected from the group consisting of capsules, tablets, dragees, pills, lozenges, powders and granules.

Oral pharmaceutical compositions according to this invention may be formulated as mutliparticulate dosage forms.

Oral pharmaceutical compositions according to this invention may be formulated as mutliparticulate dosage forms and may be selected from the group consisting of pellets, microparticles, nanoparticles, liquid or semisolid fill formulations in soft- or hard capsules, enteric coated soft-hard capsules.

In one embodiment the oral pharmaceutical compositions may be prepared with one or more coatings such as enteric coatings or be formulated as delayed release formulations according to methods well known in the art.

Enteric or delayed release coatings according to this invention may be based on poly(meth)acrylates commercially known as Eudragit®.

In one embodiment, the pharmaceutical composition according to the invention is used for the preparation of a medicament.

In one embodiment, the pharmaceutical composition according to the invention is used for the preparation of a medicament for the treatment or prevention of hyperglycemia, type 2 diabetes mellitus, impaired glucose tolerance, type 1 diabetes mellitus and/or anti obesity treatment.

The terms “fatty acid N-acylated amino acid” or “acylated amino acid” may be used interchangeable and refer when used herein to an amino acids that is acylated with a fatty acid at its alpha-amino group.

Amino acids exist in the stereoisomeric form of either D (dextro) or L (levo). The D and L refer to the absolute confirmation of optically active compounds. With the exception of glycine, all other amino acids are mirror images that can not be superimposed. Most of the amino acids found in nature are of the L-type. Hence, eukaryotic proteins are always composed of L-amino acids although D-amino acids are found in bacterial cell walls and in some peptide antibiotics. At least 300 amino acids have been described in nature but only twenty of these are typically found as components in human peptides and proteins. Twenty standards amino acids are used by cells in peptide biosynthesis, and these are specified by the general genetic code. The twenty standard amino acids are Alanine (Ala), Valine (Val), Leucine (Leu), Isoleucine (Ile), Phenylalanine (Phe), Tryptophane (Trp), Methionine (Met), Proline (Pro), Apartic acid (Asp), Gltamic acid (Glu), Glycine (Gly), Serine (Ser), Threonine (Thr), Cysteine (Cys), Tyrosine (Tyr), Apsaragine (Asn), Glutamine (Gln), Lysine (Lys), Arginine (Arg) and Histidine (His).

The amino acid moiety of the modified FA-aa may be in the form of a pure enantiomer wherein the stereo configuration of the chiral amino acid moiety is either D or L (or if using the R/S terminology: either R or S) or it may be in the form of a mixture of enantiomers (D and L/R and S).

In one embodiment of the invention the amino acid moiety is in the form of a mixture of enantiomers.

In one embodiment the amino moiety is in the form of a pure enantiomer. In one embodiment the chiral amino acid moiety is in the L form. In one embodiment the chiral amino acid moiety is in the D form.

As used herein the term “non-cationic amino acid” shall be understood as referring to any amino acid selected from the group consisting of nonpolar hydrophobic amino acids, polar uncharged amino acids and polar acidic amino acids.

The term “nonpolar hydrophobic amino acids” as used herein refer to catogorisation of amino acids used by the person skilled in the art. The term “polar uncharged amino acids” as used herein refer to catogorisation of amino acids used by the person skilled in the art. The term “and polar acidic amino acids” as used herein refer to catogorisation of amino acids used by the person skilled in the art. As used herein the term “non-cationic amino acid” comprises the following amino acids: Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).

With the term “oral bioavailability” is herein meant the fraction of the administered dose of drug that reaches the systemic circulation after having been administered orally. By definition, when a medication is administered intravenously, its bioavailability is 100%. However, when a drug is administered orally the bioavailability of the active ingredient decreases due to incomplete absorption and first-pass metabolism. The biological activity of an insulin peptide may be measured in an assay as known by a person skilled in the art as e.g. described in WO 2005012347.

The term “surfactant” as used herein refers to any substance, in particular a detergent, that can adsorb at surfaces and interfaces, such as but not limited to liquid to air, liquid to liquid, liquid to container or liquid to any solid and which has no charged groups in its hydrophilic groups.

The term “permeation enhancer” when used herein refers to biologicals or chemicals that promote the absorption of drugs.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “macromolecular” or “macromolecule” used herein refer to non-polymeric molecules and comprises nucleic acids, peptides, proteins, carbohydrates, and lipids.

The term “polypeptide” and “peptide” as used herein means a compound composed of at least two constituent amino acids connected by peptide bonds. The constituent amino acids may be from the group of the amino acids encoded by the genetic code and they may be natural amino acids which are not encoded by the genetic code, as well as synthetic amino acids. Commonly known natural amino acids which are not encoded by the genetic code are e.g., γ-carboxyglutamate, ornithine, phosphoserine, D-alanine and D-glutamine. Commonly known synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid, anthranilic acid.

The term “Protein” as used herein means a biochemical compound consisting of one or more polypeptides.

The term “macromolecular therapeutic” or “therapeutic macromolecule” may be used interchangeable and as used herein refer to nucleic acids, peptides, proteins, carbohydrates, and lipids as well as non-polymeric molecules with large molecular mass used in therapy and includes without being limited thereto insulin, insulin analouges and insulin derivatives. In one embodiment large molecular mass means a molecular mass above 1500 Da. In one embodiment large molecular mass means a molecular mass between 150 Da and 6000 Da.

The term “drug”, “therapeutic”, “medicament” or “medicine” when used herein refer to an active ingredient used in a pharmaceutical composition, which may be used in therapy and thus also refer to what was defined as “macromolecular therapeutic” or “therapeutic macromolecule” in the present patent application.

With “insulin peptide”, “an insulin peptide” or “the insulin peptide” as used herein is meant human insulin comprising disulfide bridges between CysA7 and CysB7 and between CysA20 and CysB19 and an internal disulfide bridge between CysA6 and CysA11 or an insulin analogue or derivative thereof.

The term “peptide” as used herein comprises also peptides, proteins, conjugates of such peptides and proteins and biologically active fragments thereof. The term “protein” comprises peptides and also refers to proteins and biologically active fragments thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

The term “insulin” as used herein is, if not specified further, an insulin selected from the group consisting of human insulin, insulin analogues and insulin derivatives.

An insulin analogue as used herein is a polypeptide, such as a insulin peptide which has a molecular structure which formally may be derived from the structure of a naturally occurring insulin, for example that of human insulin, by deleting and/or substituting at least one amino acid residue occurring in the natural insulin and/or by adding at least one amino acid residue.

The term “insulin analogue” as used herein means a modified insulin wherein one or more amino acid residues of the insulin have been substituted by other amino acid residues and/or wherein one or more amino acid residues have been deleted from the insulin and/or wherein one or more amino acid residues have been added and/or inserted to the insulin.

In one embodiment an insulin analogue according to the invention comprises less than 8 modifications (substitutions, deletions, additions) relative to human insulin.

In one embodiment an insulin analogue comprises less than 7 modifications (substitutions, deletions, additions) relative to human insulin. In one embodiment an insulin analogue comprises less than 6 modifications (substitutions, deletions, additions) relative to human insulin.

In one embodiment an insulin analogue comprises less than 5 modifications (substitutions, deletions, additions) relative to human insulin. In one embodiment an insulin analogue comprises less than 4 modifications (substitutions, deletions, additions) relative to human insulin. In one embodiment an insulin analogue comprises less than 3 modifications (substitutions, deletions, additions) relative to human insulin. In one embodiment an insulin analogue comprises less than 2 modifications (substitutions, deletions, additions) relative to human insulin.

The term “insulin derivative” as used herein refers to chemically modified parent insulin or an analogue thereof, wherein the modification(s) are in the form of attachment of amides, carbohydrates, alkyl groups, acyl groups, esters, PEGylations, and the like.

An insulin derivative according to the invention is a naturally occurring insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin.

Herein, the term “acylated insulin” covers modification of insulin by attachment of one or more lipophilic substituents optionally via a linker to the insulin peptide.

An insulin derivative is thus human insulin, an insulin analogue or insulin peptide which comprises at least one covalent modification such as a side-chain attached to one or more amino acids of the insulin peptide.

Herein, the naming of the insulin peptide is done according to the following principles: The names are given as mutations and modifications (acylations) relative to human insulin. For the naming of the acyl moiety, the naming is done according to IUPAC nomenclature and in other cases as peptide nomenclature. For example, naming the acyl moiety:

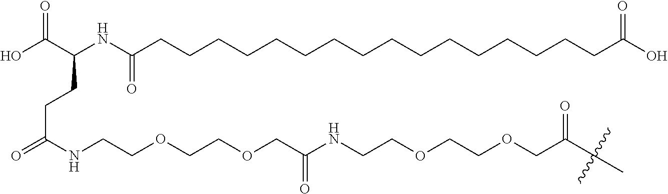 [SMILES: *C(=O)COCCOCCNC(=O)COCCOCCNC(=O)CC[C@H](NC(=O)CCCCCCCCCCCCCCCCC(=O)O)C(=O)O]

May be e.g. “octadecanedioyl-γ-L-Glu-OEG-OEG”, or “17-carboxyheptadecanoyl-γ-L-Glu-OEG-OEG”, wherein OEG is short hand notation for the amino acid —NH(CH₂)₂O(CH₂)₂OCH₂CO—, and γ-L-Glu (or g-L-Glu) is short hand notation for the L-form of the amino acid gamma glutamic acid moiety.

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enantiomers (D and L/R and S). In one embodiment of the invention the acyl moiety is in the form of a mixture of enantiomers. In one embodiment the acyl moiety is in the form of a pure enantiomer. In one embodiment the chiral amino acid moiety of the acyl moiety is in the L form. In one embodiment the chiral amino acid moiety of the acyl moiety is in the D form.

In one embodiment an insulin derivative in an oral pharmaceutical composition according to the invention is an insulin peptide that is acylated in one or more amino acids of the insulin peptide.

In one embodiment an insulin derivative in an oral pharmaceutical composition according to the invention is an insulin peptide that is stabilized towards proteolytic degradation (by specific mutations) and further acylated at the B29-lysine. A non-limiting example of insulin peptides that are stabilized towards proteolytic degradation (by specific mutations) may e.g. be found in WO 2008034881, which is hereby incorporated by reference.

The acylated insulin peptides suitable for this invention may be mono-substituted having only one acylation group attached to a lysine amino acid residue in the protease stabilized insulin molecule.

A non-limiting list of acylated insulin peptides suitable for the liquid oral pharmaceutical composition of the invention may e.g. be found in WO 2009/115469 such as in the passage beginning on page 24 thereof and continuing the next 6 pages.

In one embodiment of the invention, the acylated insulin peptide is selected from the group consisting of:

B29K(N(ε)hexadecanedioyl-γ-L-Glu) A14E B25H desB30 human insulin;

B29K(N(ε)octadecanedioyl-γ-L-Glu-OEG-OEG) desB30 human insulin;

B29K(N(ε)octadecanedioyl-γ-L-Glu) A14E B25H desB30 human insulin;

B29K(N(ε)eicosanedioyl-γ-L-Glu) A14E B25H desB30 human insulin;

B29K(N(ε)octadecanedioyl-γ-L-Glu-OEG-OEG) A14E B25H desB30 human insulin;

B29K(N(ε)eicosanedioyl-γ-L-Glu-OEG-OEG) A14E B25H desB30 human insulin;

B29K(N(ε)eicosanedioyl-γ-L-Glu-OEG-OEG) A14E B16H B25H desB30 human insulin;

B29K(N(ε)hexadecanedioyl-γ-L-Glu) A14E B16H B25H desB30 human insulin;

B29K(N(ε)eicosanedioyl-γ-L-Glu-OEG-OEG) A14E B16H B25H desB30 human insulin; and

B29K(N(ε)octadecanedioyl) A14E B25H desB30 human insulin.

In one embodiment of the invention, the insulin derivative is B29K(N(ε)octadecanedioyl-γ-L-Glu-OEG-OEG) A14E B25H desB30 human insulin.

A non-limiting list of acylated insulin peptides suitable for the liquid oral pharmaceutical composition of the invention may e.g. be found in the PCT application WO2011068019 such as outlined and exemplified in but not limited to the passage beginning on page 20 line 20 and continuing the next 6 pages, to be published in April 2013.

In one embodiment of the invention, the acylated insulin peptide is selected from the group consisting of N-terminally modified insulin consisting of:

A1(N^{α},N^{α}-Dimethyl), A14E, B1(N^{α},N^{α}-dimethyl), B25H, B29K(N^{ε}octadecanedioyl-gGlu-2×OEG), desB30 human insulin

A1(N^{α},N^{α}-Diethyl), A14E, B1(N^{α},N^{α}-diethyl), B25H, B29K(N^{ε}Octadecanedioyl-gGlu-2×OEG), desB30 human insulin

A1(N^{α},N^{α}-Dimethyl), A14E, B1(N^{α},N^{α}-dimethyl), B16H, B25H, B29K(N^{ε}hexadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu-2×OEG), desB30 human insulin

A1(N^{α},N^{α}-Dimethyl), A14E, B1(N^{α},N^{α}-dimethyl), desB27, B29K(N^{ε}octadecanedioyl-gGlu-2×OEG), desB30 human insulin

A1(N^{α},N^{α}-Dimethyl), A14E, B1(N^{α},N^{α}-dimethyl), B16H, B25H, B29K(N^{ε}eicosanedioyl-gGlu-2×OEG), desB30 human insulin

A1G(N^{α},N^{α}-Dimethyl), A14E, B1F(N^{α},N^{α}-dimethyl), B25H, desB27, B29K(N^{ε}hexadecanedioyl-gGlu), desB30 human insulin

A1G(N^{α},N^{α}-Dimethyl), A14E, B1F(N(alpha),N(N^{α},N^{α}-dimethyl), B25H, desB27, B29K(N^{ε}hexadecanedioyl-gGlu-2×OEG), desB30 human insulin

A1(N^{α},N^{α}-Dimethyl), A14E, B1(N^{α},N^{α}-dimethyl), desB27, B29K(N^{ε}octadecanedioyl-gGlu), desB30 human insulin

A1(N^{α},N^{α}-Dimethyl), A14E, B1(N^{α},N^{α}-dimethyl), B25H, B29K(N^{ε}octadecanedioyl-gGlu), desB30 human insulin

A1(N^{α}Carbamoyl), A14E, B1(N^{α}Carbamoyl), B25H, B29K(N^{ε}octadecanedioyl-gGlu-2×OEG), desB30 human insulin

A1(N^{α}Carbamoyl), A14E, B1(N^{α}Carbamoyl), B25H, B29K(N^{ε}hexadecanedioyl-gGlu), desB30 human insulin

A1(N^{α}Carbamoyl), A14E, B1(N^{α}Carbamoyl), B25H, B29K(N^{ε}eicosanedioyl-gGlu), desB30 human insulin

A1(N^{α}Carbamoyl), A14E, B1(N^{α}Carbamoyl), B25H, B29K(N^{ε}eicosanedioyl-gGlu-2×OEG), desB30 human insulin

A1(N^{α}Carbamoyl), A14E, B1(N^{α}Carbamoyl), B16H, B25H, B29K(N^{ε}eicosanedioyl-gGlu-2×OEG), desB30 human insulin

A1(N^{α}Carbamoyl), A14E, B1(N^{α}Carbamoyl), B25H, desB27, B29K(N^{ε}octadecandioyl-gGlu), desB30 human insulin

A1(N^{α}Carbamoyl), A14E, B1(N^{α}Carbamoyl), B25H, desB27, B29K(N^{ε}octadecandioyl-gGlu-2×OEG), desB30 human insulin

A1G(N(alpha)carbamoyl), A14E, B1F(N(alpha)carbamoyl), desB27, B29K(N(eps)hexadecanedioyl-gGlu), desB30 human insulin

A1G(N(alpha)carbamoyl), A14E, B1F(N(alpha)carbamoyl), desB27, B29K(Neps)-hexadecanedioyl-gGlu-2×OEG), desB30 human insulin

A1G(N(alpha)carbamoyl), A14E, B1F(N(alpha)carbamoyl), desB27, B29K(Neps)-eicosanedioyl-gGlu), desB30 human insulin

A1G(N^{α}carbamoyl), A14E, B1F(N^{α}carbamoyl), B16H, desB27, B29K(Neps)-eicosanedioyl-gGlu-2×OEG), desB30 human insulin

A1(N^{α}Carbamoyl), A14E, B1(N^{α}Carbamoyl), desB27, B29K(N^{ε}octadecanedioyl-gGlu), desB30 human insulin

A1(N^{α}Carbamoyl), A14E, B1(N^{α}Carbamoyl), B16H, B25H, B29K(N^{ε}eicosanedioyl-gGlu), desB30 human insulin

A1(N^{α}Carbamoyl), A14E, B1(N^{α}Carbamoyl), desB27, B29K(N^{ε}octadecanedioyl-gGlu-2×OEG), desB30 human insulin

A1(N^{α}Carbamoyl), A14E, B1(N^{α}carbamoyl), B25H, B29K(N^{ε}octadecanedioyl-gGlu), desB30 human insulin

A1(N^{α}Carbamoyl), A14E, B1(N^{α}Carbamoyl), B16H, B25H, B29K(N^{ε}eicosanedioyl-gGlu), desB30 human insulin

A1G(N^{α}carbamoyl), A14E, B1F(N^{α}carbamoyl), B25H, desB27, B29K(N^{ε}eicosanedioyl-gGlu-2×OEG), desB30 human insulin

A1G(N^{α}carbamoyl), A14E, B1F(N^{α}carbamoyl), desB27, B29K(N^{ε}eicosanedioyl-gGlu-2×OEG), desB30 human insulin

A1G(N^{α}carbamoyl), A14E, B1F(N^{α}carbamoyl), B16H, desB27, B29K(N^{ε}-eicosanedioyl-gGlu-2×OEG), desB30 human insulin

A1G(N^{α}thiocarbamoyl), A14E, B1F(N N^{α}thiocarbamoyl), B25H, desB27, B29K(N^{ε}-octadecanedioyl-gGlu-2×OEG), desB30 human insulin

A1(N^{α}Acetyl), A14E, B1(N^{α}Acetyl), B25H, B29K(N^{ε}hexadecanedioyl-gGlu), desB30 human insulin

A1(N^{α}Acetyl), A14E, B1(N^{α}Acetyl), B25H, desB27, B29K(N^{ε}octadecanedioyl-gGlu), desB30 human insulin

A1(N^{α}Acetyl), A14E, B1(N^{α}Acetyl), B25H, B29K(N^{ε}octadecandioyl-gGlu-2×OEG), desB30 human insulin

A1(N^{α}Dimethylglycyl), A14E, B1(N^{α}Dimethylglycyl), B25H, B29K(N^{ε}octadecanedioyl-gGlu-2×OEG), desB30 human insulin

A1(N^{α}3-(N,N-Dimethylamino)propionyl), A14E, B1 (N^{α}3-(N,N-dimethylamino)propionyl), B25H, B29K(N^{ε}octadecanedioyl-gGlu-2×OEG), desB30 human insulin

A1(N^{α}4-(N,N-Dimethylamino)butanoyl), A14E, B1 (N^{α}4-(N,N-dimethylamino)butanoyl), B25H, B29K(N^{ε}octadecanedioyl-gGlu-2×OEG), desB30 human insulin

A1(N^{α}3-(1-Piperidinyl)propionyl), A14E, B1(N^{α}3-(1-piperidinyl)propionyl), B25H, B29K(N^{ε}octadecanedioyl-gGlu-2×OEG), desB30 human insulin

A1(N^{α}Dimethylglycyl), A14E, B1(N^{α}Dimethylglycyl), B25H, desB27, B29K(N^{ε}octadecanedioyl-gGlu), desB30 human insulin

A1G(N^{α}acetyl), A14E, B1F(N^{α}acetyl), B25H, desB27, B29K(N^{ε}octadecanedioyl-gGlu-2×OEG), desB30 human insulin

A1G(N^{α}2-Picolyl), A14E, B1F(N^{α}2-Picolyl), B25H, desB27, B29K(N(eps)-octadecanedioyl-gGlu-2×OEG), desB30 human insulin

A1(N^{α}Acetyl), A14E, B1(N^{α}Acetyl), B25H, B29K(N^{ε}eicosanedioyl-gGlu), desB30 human insulin

A1(N^{α}Acetyl), A14E, B1(N^{α}Acetyl), B25H, B29K(N^{ε}eicosanedioyl-gGlu-2×OEG), desB30 human insulin

A1(N^{α}Acetyl), A14E, B1(N^{α}Acetyl), B16H, B25H, B29K(N^{ε}eicosanedioyl-gGlu-2×OEG), desB30 human insulin

A1(N^{α}Acetyl), A14E, B1(N^{α}Acetyl), B16H, B25H, B29K(N^{ε}eicosanedioyl-gGlu), desB30 human insulin

A1(N^{α}Dimethylglycyl), A14E, B1(N^{α}Dimethylglycyl), B16H, B25H, B29K(N^{ε}hexadecanedioyl-gGlu), desB30 human insulin

A-1(N^{α}Trimethyl), A14E, B-1(N^{α}Trimethyl), B25H, B29K(N^{ε}octadecanedioyl-gGlu-2×OEG), desB30 human insulin

A1(N^{α}Acetyl), A14E, B1(N^{α}Acetyl), desB27, B29K(N^{ε}octadecanedioyl-gGlu), desB30 human insulin

A1(N^{α}Acetyl), A14E, B1(N^{α}Acetyl), desB27, B29K(N^{ε}octadecanedioyl-gGlu-2×OEG), desB30 human insulin

A1(N^{α}Acetyl), A14E, B1(N^{α}Acetyl), B25H, B29K(N^{ε}octadecanedioyl-gGlu), desB30 human insulin

A1G(N^{α}Acetyl), A14E, B1F(N^{α}Acetyl), desB27, B29K(N^{ε}eicosanedioyl-gGlu), desB30 human insulin

A1G(N^{α}Acetyl), A14E, B1F(N^{α}Acetyl), desB27, B29K(N^{ε}eicosanedioyl-gGlu-2×OEG), desB30 human insulin

A1G(N^{α}Acetyl), A14E, B1F(N^{α}Acetyl), B25H, desB27, B29K(N^{ε}eicosanedioyl-gGlu-2×OEG), desB30 human insulin

A1(N^{α}Succinyl), A14E, B1(N^{α}succinyl), B25H, desB27, B29K(N^{ε}octadecanedioyl-gGlu-2×OEG), desB30 human insulin

A1(N^{α}Succinyl), A14E, B1(N^{α}succinyl), B25H, B29K(N^{ε}octadecanedioyl-gGlu-2×OEG), desB30 human insulin

A1(N^{α}Succinyl), A14E, B1(N^{α}succinyl), desB27, B29K(N^{ε}octadecanedioyl-gGlu-2×OEG), desB30 human insulin

A1(N^{α}Glutaryl), A14E, B1(N^{α}glutaryl), B25H, B29K(N^{ε}octadecanedioyl-gGlu-2×OEG), desB30 human insulin

A1(N^{α}Glutaryl), A14E, B1(N^{α}glutaryl), desB27, B29K(N^{ε}octadecanedioyl-gGlu-2×OEG), desB30 human insulin

A1(N^{α}Diglycolyl), A14E, B1(N^{α}diglycolyl), B25H, desB27, B29K(N^{ε}octadecanedioyl-gGlu-2×OEG), desB30 human insulin

A1(N^{α}Glutaryl), A14E, B1(N^{α}glutaryl), B25H, desB27, B29K(N^{ε}octadecanedioyl-gGlu-2×OEG), desB30 human insulin

A1(N^{α}Succinyl), A14E, B1(N^{α}succinyl), desB27, B29K(N^{ε}octadecanedioyl-gGlu), desB30 human insulin

A1(N^{α}Succinyl), A14E, B1(N^{α}succinyl), B25H, desB27, B29K(N^{ε}eicosanedioyl-gGlu-2×OEG), desB30 human insulin

A1(N^{α}Succinyl), A14E, B1(N^{α}succinyl), desB27, B29K(N^{ε}eicosanedioyl-gGlu-2×OEG), desB30 human insulin

A1(N^{α}Succinyl), A14E, B1(N^{α}succinyl), B16H, desB27, B29K(N^{ε}eicosanedioyl-gGlu-2×OEG), desB30 human insulin

A1(N^{α}Succinyl), A14E, B1(N^{α}succinyl), B25H, B29K(N^{ε}eicosanedioyl-gGlu-2×OEG), desB30 human insulin

A1(N^{α}Succinyl), A14E, B1(N^{α}succinyl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu-2×OEG), desB30 human insulin

A1(N^{α}Glutaryl), A14E, B1(N^{α}glutaryl), B25H, desB27, B29K(N^{ε}eicosanedioyl-gGlu-2×OEG), desB30 human insulin

A1(N^{α}Glutaryl), A14E, B1(N^{α}glutaryl), desB27, B29K(N^{ε}eicosanedioyl-gGlu-2×OEG), desB30 human insulin

A1(N^{α}Glutaryl), A14E, B1(N^{α}glutaryl), B25H, B29K(N^{ε}eicosanedioyl-gGlu-2×OEG), desB30 human insulin

In one embodiment, an N-terminally modified insulin according to the invention has a peptide part which is selected from the group consisting of the following insulin peptides (i.e. insulins of the invention without N-terminal modifications and without the “lipophilic substituent” or acyl moiety): A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H, A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(0)P, A14E, B25H, desB30 human insulin; A14E, B(−1)P, B(0)P, B25H, desB30 human insulin; A(−1)P, A(0)P, A14E, B(−1)P, B(0)P, B25H, desB30 human insulin; A14E, B25H, B30T, B31L, B32E human insulin; A14E, B25H human insulin; A14E, B16H, B25H, desB30 human insulin; A14E, B10P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, A21G, B25H, B26G, B27G, B28G, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H, A14E, B1E, B25H, desB30 human insulin; A13N, A14E, B25H, desB30 human insulin; A13N, A14E, B1E, B25H, desB30 human insulin; A(−2)G, A(−1)P, A(0)P, A14E, B25H, desB30 human insulin; A14E, B(−2)G, B(−1)P, B(0)P, B25H, desB30 human insulin; A(−2)G, A(−1)P, A(0)P, A14E, B(−2)G, B(−1)P, B(0)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, desB27, desB30 human insulin; A14H, desB27, desB30 human insulin; A14E, B16H, desB27, desB30 human insulin; A14H, B16H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N, A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, B16R, B25H, desB30 human insulin; A14E, B16D, B25H, desB30 human insulin; A14E, B16Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, B10D, B25H human insulin; and A8H, A14E, B10E, B25H, desB30 human insulin and this embodiment may, optionally, comprise B25H, desB30 human insulin and B25N, desB30 human insulin.

In a preferred embodiment, a N-terminally modified insulin according to the invention has a peptide part which is selected from the group consisting of: A14E, B25H, desB30 human insulin; A14E, B16H, B25H, desB30 human insulin; A14E, B16E, B25H, desB30 human insulin; A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.

In a preferred embodiment, a N-terminally modified insulin according to the invention has a peptide part which is selected from any one of the insulins mentioned above that, in addition, are containing the desB27 mutation.

In a preferred embodiment, a N-terminally modified insulin according to the invention has a peptide part which is selected from the group consisting of: A14E, B25H, desB27, desB30 human insulin; A14E, B16H, B25H, desB27, desB30 human insulin; A14E, desB27, desB30 human insulin; A14E, B16E, B25H, desB27, desB30 human insulin; and B25H, desB27, desB30 human insulin.

In one embodiment, a N-terminally modified insulin according to the invention has a peptide part which is selected from any of the above mentioned insulins and, in addition, comprise one or two of the following mutations in position A21 and/or B3 to improve chemical stability: A21G, desA21, B3Q, or B3G.

In a preferred embodiment, a N-terminally modified insulin according to the invention has a peptide part which is selected from the group consisting of: A14E, A21G, B25H, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A14E, A21G, B25H, B26G, B27G, B28G, desB30 human insulin; A21G, B25H, desB30 human insulin and A21G, B25N, desB30 human insulin, and, preferably, it is selected from the following protease stabilised insulins: A14E, A21G, B25H, desB30 human insulin; A14E, A21G, desB27, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A21G, B25H, desB30 human insulin and A21G, B25N, desB30 human insulin.

Herein, the term “acylated insulin” covers modification of insulin by attachment of one or more lipophilic substituents optionally via a linker to the insulin peptide.

A “lipophilic substituent” is herein understood as a side chain consisting of a fatty acid or a fatty diacid attached to the insulin, optionally via a linker, in an amino acid position such as LysB29, or equivalent.

The insulin peptide may be present in an amount of a pharmaceutical composition according to the invention in up to about 20% such as up to about 10% by weight of the total pharmaceutical composition, or from about 0.1% such as from about 1%. In one embodiment of the invention, the insulin peptide is present in an amount from about 0.1% to about 20%, in a further embodiment from about 0.1% to 15%, 0.1% to 10%, 1% to 8% or from about 1% to 5% by weight of the total composition. It is intended, however, that the choice of a particular level of insulin peptide will be made in accordance with factors well-known in the pharmaceutical arts, including the solubility of the insulin peptide in the polar organic solvent or optional hydrophilic component or surfactant used, or a mixture thereof, mode of administration and the size and condition of the patient.

Each unit dosage will suitably contain from 1 mg to 200 mg insulin peptide, e.g. about 1 mg, 5 mg, 10 mg, 15 mg, 25 mg, 50 mg, 80 mg, 90 mg, 100 mg, 150 mg, 200 mg insulin peptide, e.g. between 5 mg and 200 mg of insulin peptide. In one embodiment of the invention each unit dosage contains between 10 mg and 200 mg of insulin peptide. In a further embodiment a unit dosage form contains between 10 mg and 100 mg of insulin peptide.

One embodiment of the invention, the unit dosage form contains between 20 mg and 80 mg of insulin peptide. In yet a further embodiment of the invention, the unit dosage form contains between 30 mg and 60 mg of insulin peptide.

In one embodiment of the invention, the unit dosage form contains between 30 mg and 50 mg of insulin peptide. Such unit dosage forms are suitable for administration 1-5 times daily depending upon the particular purpose of therapy.

The production of polypeptides and peptides such as insulin is well known in the art. Polypeptides or peptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides or peptides may also be produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants.

As used herein, the term “microemulsion preconcentrate” means a composition, which spontaneously forms a microemulsion or a nanoemulsion, e.g., an oil-in-water microemulsion or nanoemulsion, swollen micelle, micellar solution, in an aqueous medium, e.g. in water or in the gastrointestinal fluids after oral application. The composition self-emulsifies upon dilution in an aqueous medium for example in a dilution of 1:5, 1:10, 1:50, 1:100 or higher. In one embodiment the composition according to the present invention forms the microemulsion or nanoemulsion comprising particles or domains of a size below 100 nm in diameter. The term “domain size” or “particle size” as used herein refers to repetitive scattering units and may be measured by e.g., small angle X-ray. In one embodiment of the invention, the domain size is smaller than 150 nm, in another embodiment, smaller than 100 nm and in another embodiment, smaller than 50 nm, in another embodiment, smaller than 20 nm, in another embodiment, smaller than 15 nm, in yet another embodiment, smaller than 10 nm.

“SEDDS” (self emulsifying drug delivery systems) are herein defined as mixtures of a hydrophilic component, a surfactant, optionally a co-surfactant or lipid component and a therapeutic macromolecule that forms spontaneously a fine oil in water emulsion when exposed to aqueous media under conditions of gentle agitation or digestive motility that would be encountered in the GI tract. “SMEDDS” (self micro-emulsifying drug delivery systems) are herein defined as isotropic mixtures of a hydrophilic component a surfactant, optionally a co-surfactant or lipid component and a therapeutic macromolecule that rapidly form an oil in water microemulsion or nanoemulsion when exposed to aqueous media under conditions of gentle agitation or digestive motility that would be encountered in the GI tract. “SNEDDS” (self nano-emulsifying drug delivery systems) are herein defined as isotropic mixtures of a hydrophilic component, at least one surfactant with HLB above 10, optionally a co-surfactant and optionally a lipid component and a therapeutic macromolecule that rapidly form a nanoemulsion (droplet size below 20 nm in diameter as e.g. measured by PCS) when exposed to aqueous media under conditions of gentle agitation or digestive motility that would be encountered in the GI tract.

As used herein, the term “emulsion” refers to a slightly opaque, opalescent or opague colloidal coarse dispersion that is formed spontaneously or substantially spontaneously when its components are brought into contact with an aqueous medium.

As used herein, the term “microemulsion” refers to a clear or translucent, slightly opaque, opalescent, non-opaque or substantially non-opaque colloidal dispersion that is formed spontaneously or substantially spontaneously when its components are brought into contact with an aqueous medium.

A microemulsion is thermodynamically stable and contains homogenously dispersed particles or domains, for example of a solid or liquid state (e.g., liquid lipid particles or droplets), of a mean diameter of less than 150 nm as measured by standard light scattering techniques, e.g., using a MALVERN ZETASIZER Nano ZS. In one embodiment when the pharmaceutical composition according to the invention is brought into contact with an aqueous medium a microemulsion is formed which contains homogenously dispersed particles or domains of a mean diameter of less than 100 nm, such as less than 50 nm, less than 40 nm and less than 30 nm. Thus, the term “Z average (nm)” refers to the partice size of the particles or domains of said microemulsion. The term “PDI” is the abbreviation of the term “polydispersity index” and is a measure of the heterogeneity of sizes of molecules or particles in a mixture.

The term “domain size” as used herein refers to repetitive scattering units and may be measured by e.g., small angle X-ray. In one embodiment of the invention, the domain size is smaller than 150 nm, In one embodiment, smaller than 100 nm and In one embodiment, smaller than 50 nm, In one embodiment, smaller than 20 nm, In one embodiment, smaller than 15 nm, in yet another embodiment, smaller than 10 nm.

As used herein, the term “nanoemulsion” refers to a clear or translucent, slightly opaque, opalescent, non-opaque or substantially non-opaque colloidal dispersion with particle or droplet size below 20 nm in diameter (as e.g. measured by PCS) that is formed spontaneously or substantially spontaneously when its components are brought into contact with an aqueous medium. In one embodiment when the pharmaceutical composition according to the invention is brought into contact with an aqueous medium a microemulsion is formed which contains homogenously dispersed particles or domains of a mean diameter of less than 20 nm, such as less than 15 nm, less than 10 nm and greater than about 2-4 nm.

As used herein the term “spontaneously dispersible” when referring to a preconcentrate refers to a composition that is capable of producing colloidal structures such as nanoemulsions, microemulsions, emulsions and other colloidal systems, when diluted with an aqueous medium when the components of the composition of the invention are brought into contact with an aqueous medium, e.g. by simple shaking by hand for a short period of time, for example for ten seconds. In one embodiment a spontaneously dispersible concentrate according to the invention is a SEDDS, SMEDDS or SNEDDS.

The term “non-ionic surfactant” as used herein refers to any substance, in particular a detergent, that can adsorb at surfaces and interfaces, like liquid to air, liquid to liquid, liquid to container or liquid to any solid and which has no charged groups in its hydrophilic group(s) (sometimes referred to as “heads”). The non-ionic surfactant may be selected from a detergent such as ethoxylated castor oil, polyglycolyzed glycerides, acetylated monoglycerides and sorbitan fatty acid esters, polysorbate such as polysorbate-20, polysorbate-40, polysorbate-60, polysorbate-80, super refined polysorbate 20, super refined polysorbate 40, super refined polysorbate 60 and super refined polysorbate 80 (where the term “super refined” is used by the supplier Croda for their high purity Tween products), poloxamers such as poloxamer 188 and poloxamer 407, polyoxyethylene sorbitan fatty acid esters, polyoxyethylene derivatives such as alkylated and alkoxylated derivatives (Tweens, e.g. Tween-20 or Tween-80), block copolymers such as polyethyleneoxide/polypropyleneoxide block copolymers (e.g. Pluronics/Tetronics, Triton X-100 and/or Synperonic PE/L 44 PEL) and ethoxylated sorbitan alkanoates surfactants (e.g. Tween-20, Tween-40, Tween-80, Brij-35), diglycerol laurate, diglycerol caprate, diglycerol caprylate, diglycerol monocaprylate, polyglycerol laurate, polyglycerol caprate and polyglycerol caprylate.

The term “non-aqueous” as used herein refers to a composition to which no water is added during preparation of the pharmaceutical composition. It is known to the person skilled in the art that a composition which has been prepared without addition of water may take up small amounts of water from the surroundings during handling of the pharmaceutical composition such as e.g. a soft-capsule or a hard-capsule used to encapsulate the composition. Also, the insulin peptide and/or one or more of the excipients in the pharmaceutical composition may have small amounts of water bound to it before preparing a pharmaceutical composition according to the invention. A non-aqueous pharmaceutical composition according to the invention may thus contain small amounts of water. In one embodiment a non-aqueous pharmaceutical composition according to the invention comprises less than 10% (w/w) water. In another embodiment, the composition according to the invention comprises less than 5% (w/w) water. In another embodiment, the composition according to the invention comprises less than 4% (w/w) water, in another embodiment less than 3% (w/w) water, in another embodiment less than 2% (w/w) water and in yet another embodiment less than 1% (w/w) water. In one embodiment the composition accord 0% (w/w) water

Examples of other non-ionic surfactants include, but are not limited to:

1. Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially available, e.g., the CREMOPHOR series from BASF Corp. (Mt. Olive, N.J.), such as CREMOPHOR RH 40 which is PEG40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. Polyoxyethylene fatty acid esters that include polyoxyethylene stearic acid esters, such as the MYRJ series from Uniqema e.g., MYRJ 53 having a m.p. of about 47° C. Particular compounds in the MYRJ series are, e.g., MYRJ 53 having an m.p. of about 47° C. and PEG-40-stearate available as MYRJ 52;
3. Sorbitan derivatives that include the TWEEN series from Uniqema, e.g., TWEEN 60;
4. Polyoxyethylene-polyoxypropylene co-polymers and block co-polymers or poloxamers, e.g., Pluronic F127 or Pluronic F68 from BASF or Synperonic PE/L from Croda;
5. Polyoxyethylene alkyl ethers, e.g., such as polyoxyethylene glycol ethers of C₁₂-C₁₈ alcohols, e.g., polyoxyl 10- or 20-cetyl ether or polyoxyl 23-lauryl ether, or 20-oleyl ether, or polyoxyl 10-, 20- or 100-stearyl ether, as known and commercially available as the BRIJ series from Uniqema. Particularly useful products from the BRIJ series are BRIJ 58; BRIJ 76; BRIJ 78; BRIJ 35, i.e. polyoxyl 23 lauryl ether; and BRIJ 98, i.e., polyoxyl 20 oleyl ether. These products have a m.p. between about 32° C. to about 43° C.;
6. Water-soluble tocopheryl PEG succinic acid esters available from Eastman Chemical Co. with a m.p. of about 36° C., e.g, TPGS, e.g., vitamin E TPGS.
7. PEG sterol ethers having, e.g., from 5-35 [CH₂—CH,—O] units, e.g., 20-30 units, e-g., SOLULAN C24 (Choleth-24 and Cetheth-24) from Chemron (Paso Robles, Calif.); similar products which may also be used are those which are known and commercially available as NIKKOL BPS-30 (polyethoxylated 30 phytosterol) and NIKKOL BPSH-25 (polyethoxylated 25 phytostanol) from Nikko Chemicals;
8. Polyglycerol fatty acid esters, e.g., having a range of glycerol units from 4-10, or 4, 6 or 10 glycerol units. For example, particularly suitable are deca-/hexa-/tetraglyceryl monostearate, e.g., DECAGLYN, HEXAGLYN and TETRAGLYN from Nikko Chemicals;
9. Alkylene polyol ether or ester, e.g., lauroyl macrogol-32 glycerides and/or stearoyl macrogol-32 glycerides which are GELUCIRE 44/14 and GELUCIRE 50/13 respectively;
10. Polyoxyethylene mono esters of a saturated C₁₀ to C₂₂, such as C₁₈ substituted e.g. hydroxy fatty acid; e.g. 12 hydroxy stearic acid PEG ester, e.g. of PEG about e.g. 600-900 e.g. 660 Daltons MW, e.g. SOLUTOL HS 15 from BASF (Ludwigshafen, 20 Germany). According to a BASF technical leaflet MEF 151E (1986), SOLUTOL HS 15 comprises about 70% polyethoxylated 12-hydroxystearate by weight and about 30% by weight unesterified polyethylene glycol component. It has a hydrogenation value of 90 to 110, a saponification value of 53 to 63, an acid number of maximum 1, and a maximum water content of 0.5% by weight;
11. Polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is commercially available as NIKKOL PBC 34 from Nikko Chemicals;
12. Polyethoxylated distearates, e.g. commercially available under the tradenames ATLAS G 1821 from Uniqema and NIKKOCDS-6000P from Nikko Chemicals.

When used herein the term “Hydrophilic-lipophilic balance” or “HLB” of a surfactant or lipophilic component is a measure of the degree to which it is hydrophilic or lipophilic, determined by calculating values for the different regions of the molecule, as described by Griffin (Griffin W C: “Classification of Surface-Active Agents by ‘HLB,’” Journal of the Society of Cosmetic Chemists 1 (1949): 311) or by Davies (Davies J T: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

“Non-ionic surfactants with HLB above 10” are a selection of non-ionic surfactants which have the common feature of having HLB above 10.

For exemplification, a non-limiting list of surfactants with HLB above 10 is provided below together with their HLB value:

Polyethylene glycol sorbitan monolaurate (Tween 20, Polysorbate 20, super refined polysorbate 20) with an HLB of 16.7;
Polyoxyethylene (20) sorbitan monooleate (Tween 80, Polysorbate 80, super refined polysorbate 80) with an HLB of 15;
Polyoxyethylene (20) sorbitan monopalmitate (Tween 40, Polysorbate 40, super refined polysorbate 40) with an HLB of 15.6;
Diglycerol caprylate (diglycerol monocaprylate, polyglycerol caprylate) with an HLB of 11.
Polyglycerol caprate (Rylo PG10 Pharma) with HLB of 10;
Caprylocaproyl macrogolglycerides (Labrasol, Labrasol ALF) with an HLB of 14;
Block polymers such as SYNPERONIC PE/L 44 (Poloxamer 124);
Polyoxyethylenestearate (Myrj 45, Macrogolstearate) with HLB of 11.1;
Polyoxyethylenestearate (Myrj 49, Macrogolstearate) with HLB of 15;
Polyoxyethylenestearate (Myrj 51, Macrogolstearate) with HLB of 16;
Polyoxyethylenestearate (Myrj 52, Macrogolstearate) with HLB of 16.9;
Polyoxyethylenestearate (Myrj 53, Macrogolstearate) with HLB of 17.9;
Polyoxyethylenestearate (Myrj 59, Macrogolstearate) with HLB of 18.8; and

Polyoxyethyleneglyceroltriricinoleat (Cremophor EL) with HLB of 13.3.

As used herein the term “amino acid” refers to any molecule that contains both amine and carboxyl functional groups.

The term “enteric coating” as used herein means a polymer coating that controls disintegration and release of the solid oral dosage form. The site of disintegration and release of the solid dosage form may be designed depending on the pH of the targeted area, where absorbtion of the therapeutic macromolecule (i.e. therapeutical active peptide or protein) is desired, thus does also include acid resistant protective coatings. The term includes known enteric coatings, but also any other coating with enteric properties, wherein said term “enteric properties” means properties controlling the disintegration and release of the solid oral dosage form (i.e. the oral pharmaceutical composition according to this invention).

The term “enteric soft- or hard capsule technology” when used herein means soft- or hard capsule technology comprising at least one element with enteric properties, such as at least one layer of an enteric coating. The term “delayed release coatings” as used herein means a polymer coating which releases the API in a delayed manner after oral dosing. Delayed release can be achieved by pH dependent or pH independent polymer coatings.

The term “co-surfactant” when used herein refers to an additional surfactant added to a composition or formulation, wherein a first surfactant is present.

In the present context, 1,2-propanediol and propylene glycol is used interchangeably.

The Following is a Non-Limiting List of Aspects Further Comprised within the Scope of the Invention:
1. An oral pharmaceutical composition comprising

a. at least one fatty acid acylated amino acid of the general formula:

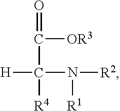

wherein R1 is a fatty acid chain comprising 8 to 18 carbon atoms, R2 is either H (i.e. hydrogen) or CH3 (i.e. methyl group), and R3 is either H, or a salt of, and R4 is a non-cationic amino acid side chain and

b. at least one therapeutic macromolecule.
2. An oral pharmaceutical composition comprising

a. at least one fatty acid acylated amino acid of the general formula:

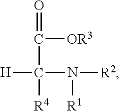

wherein R1 is a fatty acid chain comprising 8 to 18 carbon atoms,
R2 is either H (i.e. hydrogen) or CH3 (i.e. methyl group), and
R3 is either H, or a salt thereof, and
R4 is a non-cationic amino acid side chain, and

b. at least one hydrophillic peptide or protein.
3. An oral pharmaceutical composition comprising

a. at least one fatty acid acylated amino acid of the general formula:

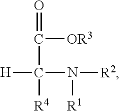

wherein R1 is a fatty acid chain comprising 8 to 18 carbon atoms,
R2 is either H (i.e. hydrogen) or CH3 (i.e. methyl group), and
R3 is either H, or a salt thereof, and
R4 is a non-cationic amino acid side chain, and

b. at least one insulin peptide.
4. An oral pharmaceutical composition according to any of the preceding aspects, wherein the amino acid residue of said at least one fatty acid acylated amino acid is based on a nonpolar hydrophobic amino acid.
5. An oral pharmaceutical composition according to any of the preceding aspects, wherein the amino acid residue of said at least one fatty acid acylated amino acid is based on a polar uncharged amino acid.
6. An oral pharmaceutical composition according to any of the preceding aspects, wherein the amino acid residue of said at least one fatty acid acylated amino acid is a based on a polar acidic amino acid.
7. A solid oral composition according to any of the preceeding aspects further comprising at least one insulin.
8. A solid oral composition according to any of the preceeding aspects further comprising an enteric or delayed release coating.
9. An oral pharmaceutical composition according to any of the preceeding aspects, wherein the fatty acid moiety of said FA-aa is in the form of its free acid or salt.
10. An oral pharmaceutical composition according to any of the preceeding aspects wherein said fatty acid moiety of the FA-aa consists of 10 carbon atoms.
11. An oral pharmaceutical composition according to any of the preceeding aspects, wherein the fatty acid moiety of said FA-aa consists of 12 carbon atoms.
12. An oral pharmaceutical composition according to any of the preceeding aspects, wherein the fatty acid moiety of said FA-aa consists of 14 carbon atoms.
13. An oral pharmaceutical composition according to any of the preceeding aspects, wherein the fatty acid moiety of said FA-aa consists of 16 carbon atoms.
14. An oral pharmaceutical composition according to any of the preceeding aspects, wherein the amino acid residue of said FA-aa is selected from the group consisting of Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).
15. An oral pharmaceutical composition according to any of the preceeding aspects, wherein the amino acid residue of said FA-aa is selected from the group consisting of the form of the free acid or salt of Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).
16. An oral pharmaceutical composition according to any of the preceeding aspects, wherein the FA-aa is selected from the group consisting of: Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine, Sodium cocoyl glycine and Sodium cocoyl glutamate.
17. An oral pharmaceutical composition according to any of the preceeding aspects, wherein the FA-aa is selected from the group consisting of: Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine and Sodium cocoyl glycine, Sodium cocoyl glutamate.
18. An oral pharmaceutical composition according to any of the preceeding aspects, further comprising propylene glycol.
19. An oral pharmaceutical composition according to any of the preceeding aspects, further comprising SEDDS, SMEDDS or SNEDDS.
20. An oral pharmaceutical composition according to any of the preceeding aspects, further comprising other pharmaceutical excipients.
21. An oral pharmaceutical composition according to any of the preceeding aspects for use as a medicament.
22. An oral pharmaceutical composition according to any of the preceeding aspects for use as a medicament for treatment of Diabetes Mellitus.
23. The pharmaceutical composition according to any of the preceding aspects, wherein said hydrophilic peptide or protein is an insulin peptide.
24. The pharmaceutical composition according to any of the preceding aspects, which comprises less than 10% (w/w) water.
25. The oral pharmaceutical composition according to any of the preceding aspects, wherein the amino acid residue of said at least one fatty acid acylated amino acid is based on a nonpolar hydrophobic amino acid, a polar uncharged amino acid or polar acidic amino acid.
26. The oral composition according to any of the preceeding aspects further comprising an enteric or delayed release coating.
27. The oral pharmaceutical composition according to any of the preceeding aspects,
wherein the fatty acid acylated amino acid is in the form of its free acid or salt.
28. The oral pharmaceutical composition according to any of the preceeding embodiments wherein said fatty acid moiety of the FA-aa consists of 8, 10 or 12.
29. The oral pharmaceutical composition according to any of the preceeding embodiments wherein said fatty acid moiety of the FA-aa consists of 14, 16 or 18 carbon atoms.
30. The oral pharmaceutical composition according to any of the preceeding embodiments wherein said fatty acid moiety of the FA-aa consists of 10, 12, 14, 16 or 18 carbon atoms.
31. The oral pharmaceutical composition according to any of the preceeding embodiments wherein said fatty acid moiety of the FA-aa consists of 10 or 12.
32. The oral pharmaceutical composition according to any of the preceeding aspects, wherein the amino acid residue of said FA-aa is selected from the group consisting of Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).
33. The oral pharmaceutical composition according to any of the preceeding aspects, wherein the fatty acid acylated amino acid is selected from the group consisting of: Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine, Sodium cocoyl glycine, Sodium cocoyl glutamate Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine and Sodium cocoyl glycine, Sodium cocoyl glutamate.
34. The oral pharmaceutical composition according to any of the preceeding aspects, further comprising propylene glycol.
35. An oral pharmaceutical composition according to any of the preceeding aspects, further comprising SEDDS, SMEDDS or SNEDDS.
36. The oral pharmaceutical composition according to any of the preceeding aspects, further comprising other pharmaceutical excipients.
37. The oral pharmaceutical composition according to any of the preceeding aspects for use as a medicament.
38. The oral pharmaceutical composition according to any of the preceeding aspects for use as a medicament for treatment of Diabetes Mellitus.
39. Use of an oral pharmaceutical composition according to any of the preceeding aspects, for increasing the bioavailability of said hydrophilic peptide or protein.
40. Use of an oral pharmaceutical composition according to any of the preceeding aspects, for increasing the bioavailability of said therapeutic macromolecule.
41. Use of an oral pharmaceutical composition according to any of the preceeding aspects, for increasing the bioavailability of said therapeutic active peptide.

### Examples of Pharmaceutical Compositions Comprising Insulin Derivative and Fatty Acid Acylated Amino Acids.

### Example 1

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of fatty acid acylated amino acids. The composition was injectioned into mid-jejunum of anaesthetized overnight fasted Sprague-Dawley rats (n=6) and the pharmacokinetic profile was retrieved from the resulting records.

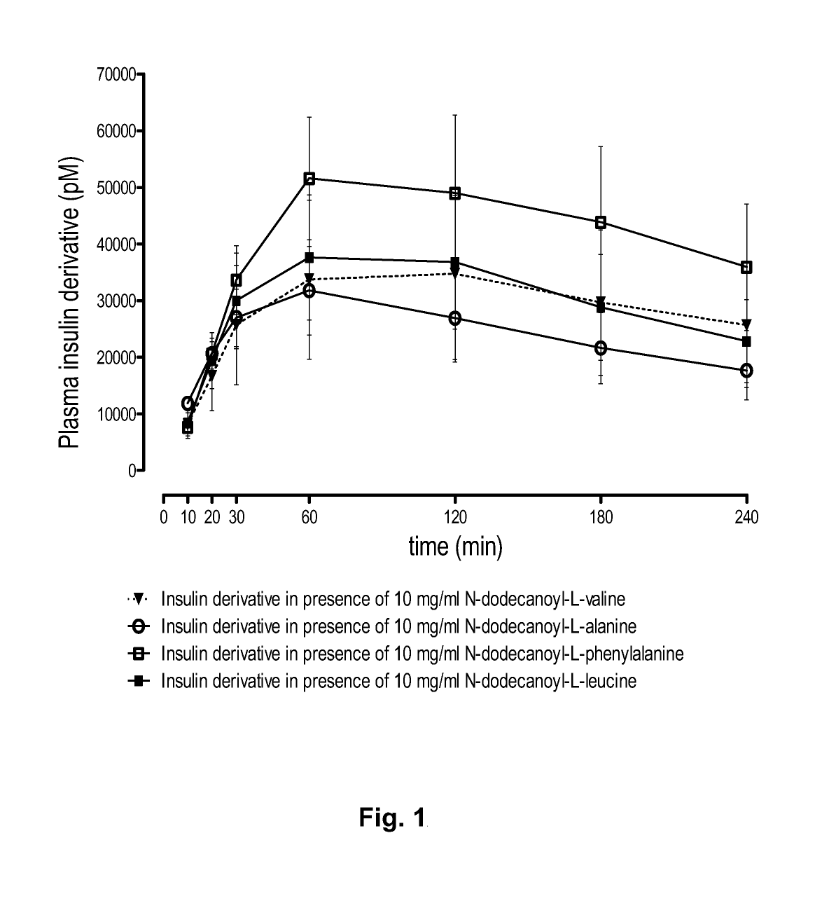

The results are shown in FIG. 1.

### Example 2

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of sodium N-capric leucine in concentrations of 10 or 20 mg/mL, respectively. The composition was injectioned into mid-jejunum of anaesthetized overnight fasted Sprague-Dawley rats (n=4-6) and the pharmacokinetic profiles were retrieved from the resulting records.

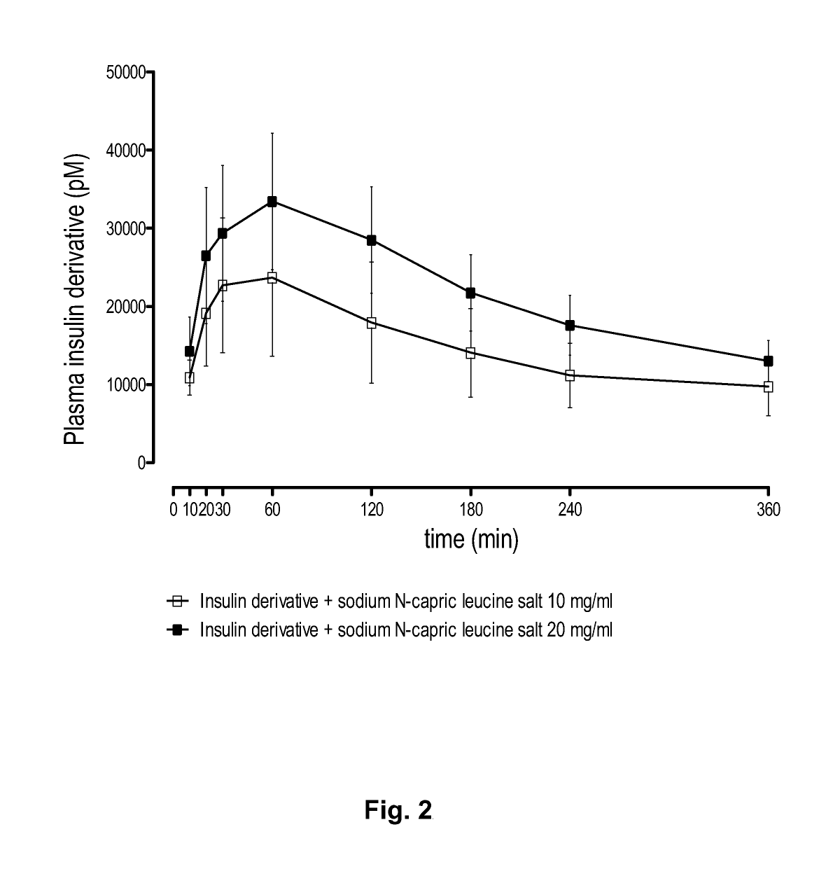

The results are shown in FIG. 2.

### Example 3

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of sodium lauroyl sarcosinate (10 m/mL) or sodium N-cocoyl sarcosinate (10 mg/mL), respectively. The formulation (-□-) with N-cocoyl sarcosine contained 50% of the co-solvent propylene glycol. The fatty acid chain distribution in the cocoyl sarcosinate was 1% C6, 8% C8, 6% 010, 48% C12, 18% C14, 8% C16, 6% C18 saturated and 5% C18 unsaturated.

The resulting compositions was injected into mid-jejunum of anaesthetized overnight fasted Sprague-Dawley rats (n=6) and the pharmacokinetic profiles.

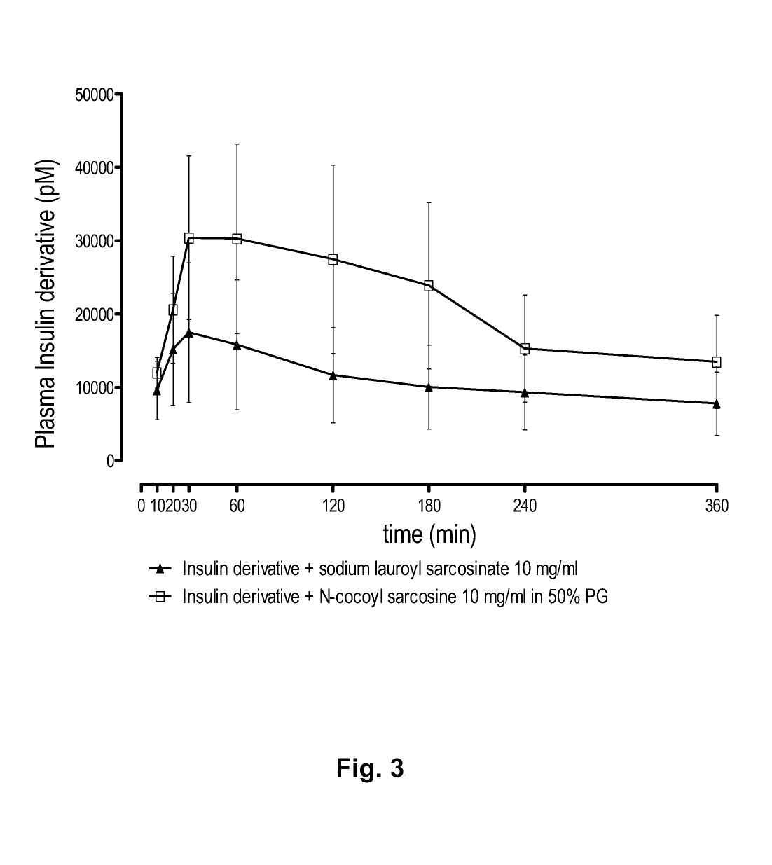

The results are shown in FIG. 3.

### Example 4

The insulin derivative A14E, B25H, B29K (N(eps)Octadecanedioyl-gGlu-OEG-OEG), desB30 human insulin (60 nmol/kg) dissolved in phosphate buffer (pH 7.4) in presence of increasing amounts (3 mg/mL, 10 mg/mL, 30 mg/mL and 100 mg/L) of sodium lauroyl sarcosinate The resulting compositions were injected into mid-jejunum of anaesthetized overnight fasted Sprague-Dawley rats (n=6) and the pharmacokinetic profiles were retrieved from the resulting records.

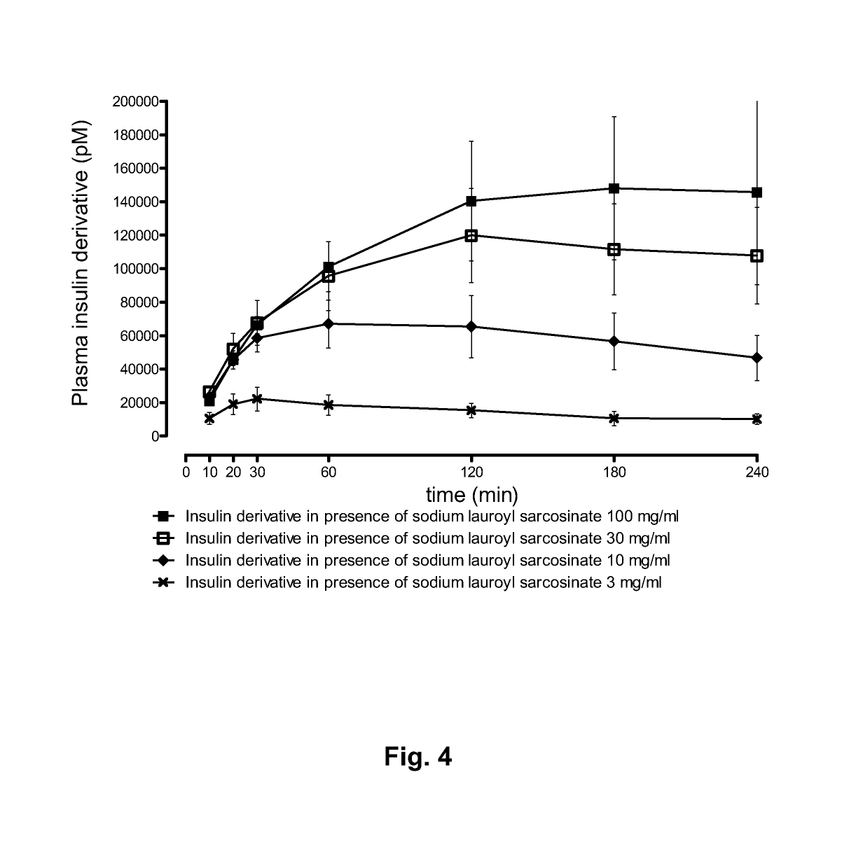

The results are shown in FIG. 4.

### Example 5

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of increasing amounts (3 mg/mL, 10 mg/mL, 30 mg/mL and 100 mg/L) of sodium myristoyl glutamate. The resulting compositions were injected into mid-jejunum of anaesthetized overnight fasted Sprague-Dawley rats (n=4-6) and the pharmacokinetic profiles were retrieved from the resulting records.

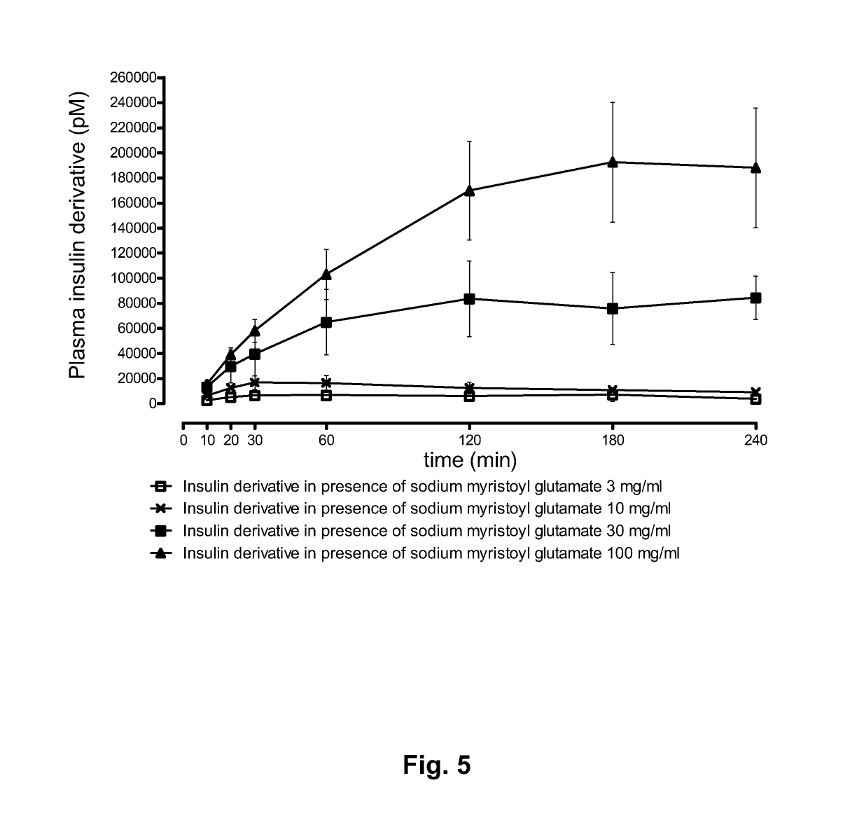

The results are shown in FIG. 5.

### Example 6

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg/mL sodium lauroyl sarcosinate. The composition was injected into colon of anaesthetized overnight fasted Sprague-Dawley rats (n=6) and the pharmacokinetic profiles were retrieved from the resulting records.

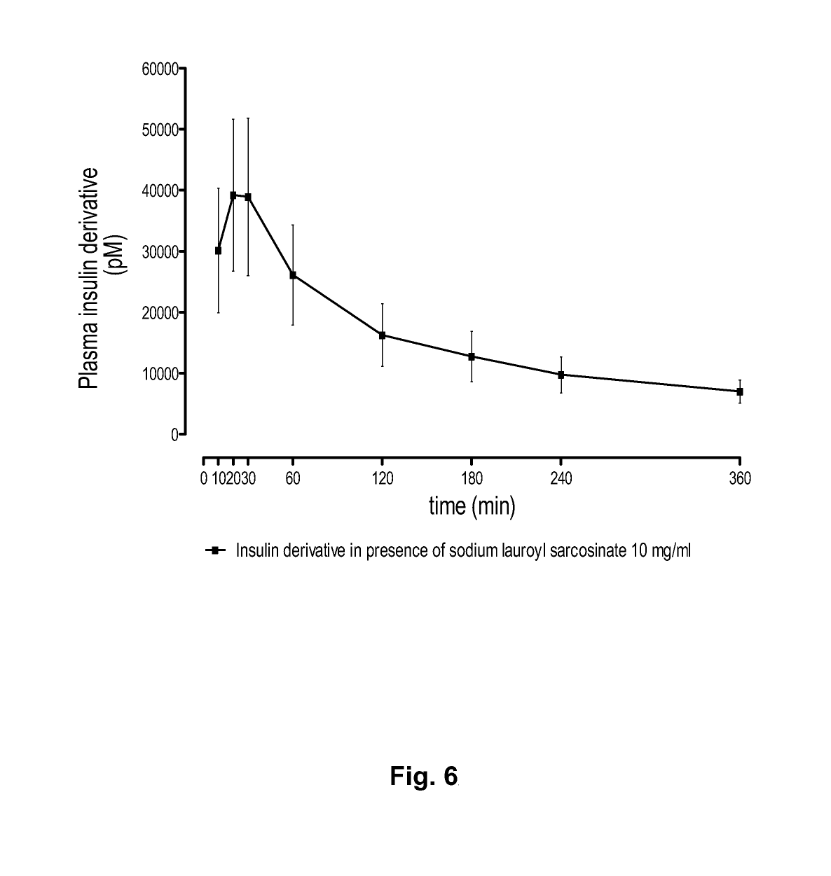

The result is shown in FIG. 6.

### Example 7

Pharmakokinetic profiles were made of the insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg/mL oleoyl sarcosinate or in presence of 10 mg/mL cocoyl sarcosinate and 16.5% of the co-solvent propylene glycol. The fatty acid chain distribution in the cocoyl sarcosinate is 1% C6, 8% C8, 6% 010, 48% C12, 18% C14, 8% C16, 6% C18 saturated and 5% C18 unsaturated.

The composition was injected into mid-jejunum of anaesthetized overnight fasted Sprague-Dawley rats (n=6) and the pharmacokinetic profiles were retrieved from the resulting records.

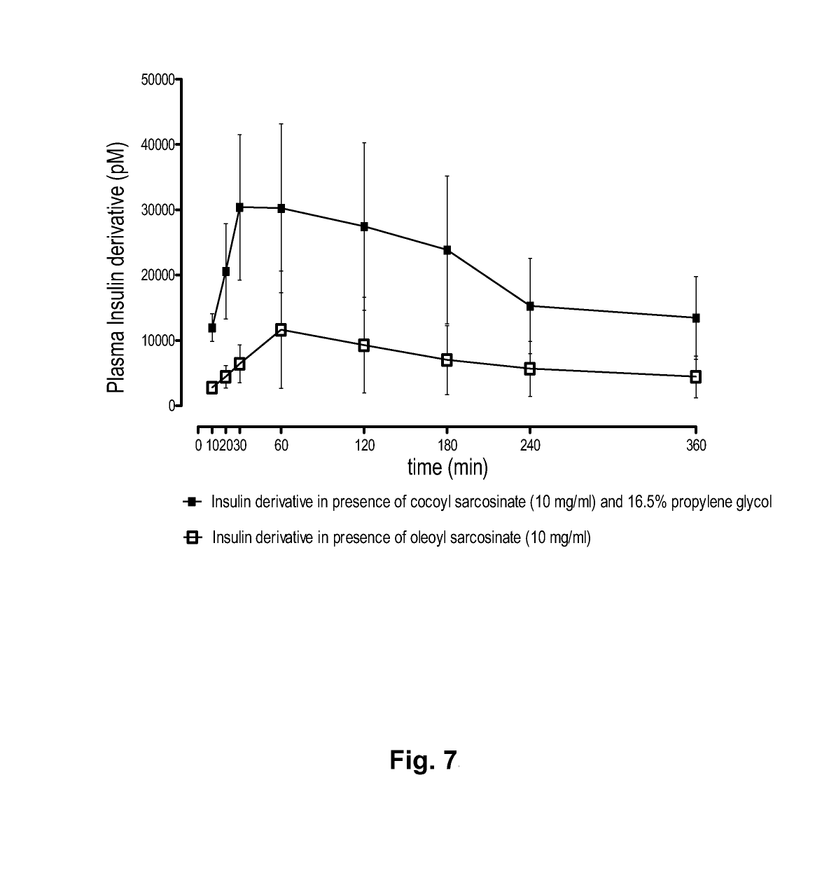

The results are shown in FIG. 7.

### Example 8

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg/mL sodium N-myristoyl-L-glutamate, sodium N-lauroyl-L-glutamate, sodium N-cocoyl-L-glutamate, sodium N-cocoyl glycinate or sodium N-steoryl-L-glutamate, respectively.

The resulting compositions were injected into mid-jejunum of anaesthetized overnight fasted Sprague-Dawley rats (n=4-6) and the pharmacokinetic profiles were retrieved from the resulting records.

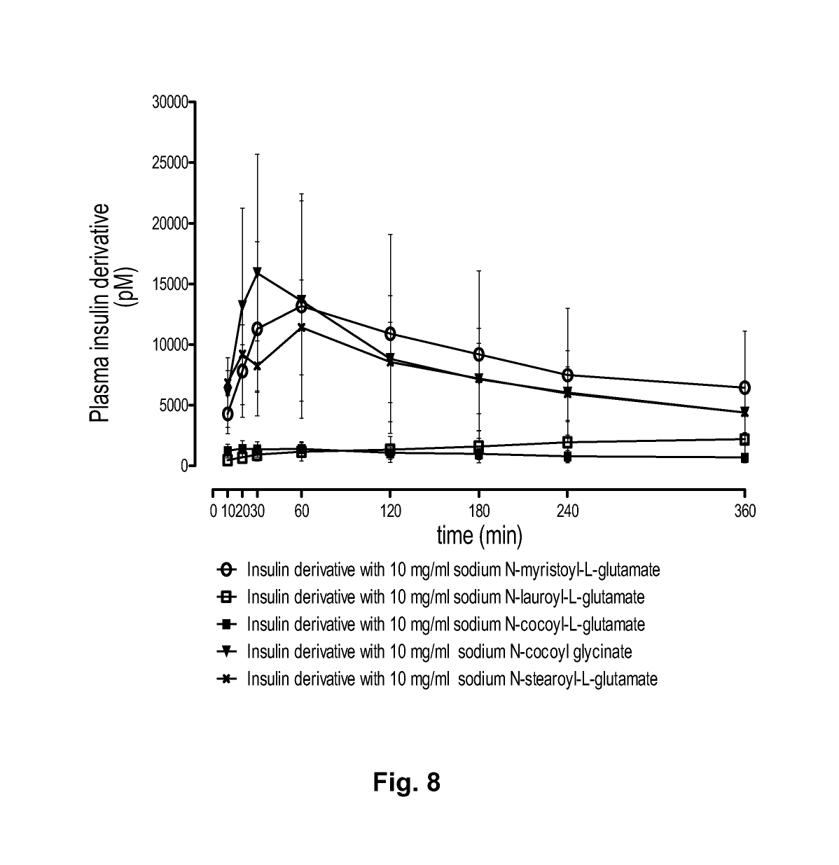

The results are shown in FIG. 8.

### Example 9

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg/mL sodium N-capric leucine, sodium, N-capric alanine, sodium N-capric phenylalanine, N-capric isoleucine, N-capric aspart, N-lauroyl leucine or N-myristoyl leucine, respectively. The resulting compositions were injected into mid-jejunum of anaesthetized overnight fasted Sprague-Dawley rats (n=5-6) and the pharmacokinetic profiles were retrieved from the resulting records.

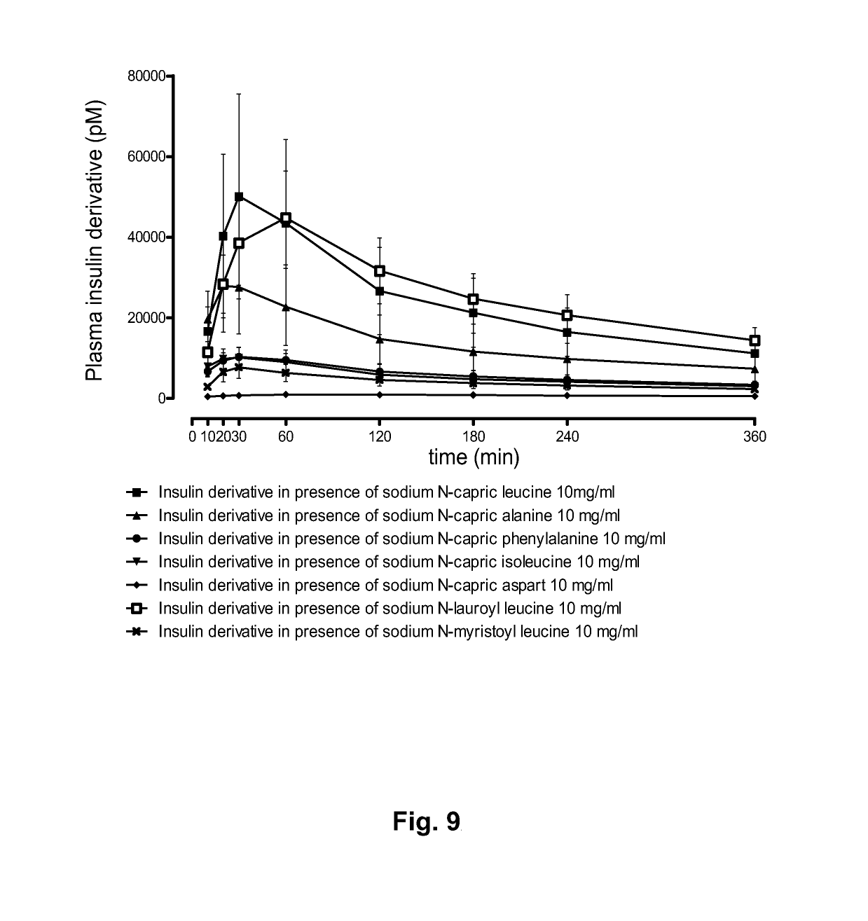

The results are shown in FIG. 9.

### Example 10

The insulin derivative A14E, B25H, B29K(N(eps)Octadecanedioyl-gGlu-OEG-OEG), desB30 human insulin (60 nmol/kg) dissolved in propylene glycol in presence of sodium N-capric leucine. The resulting composition was injected into mid-jejunum of anaesthetized overnight fasted Sprague-Dawley rats (n=6) and the pharmacokinetic profiles were retrieved from the resulting records.

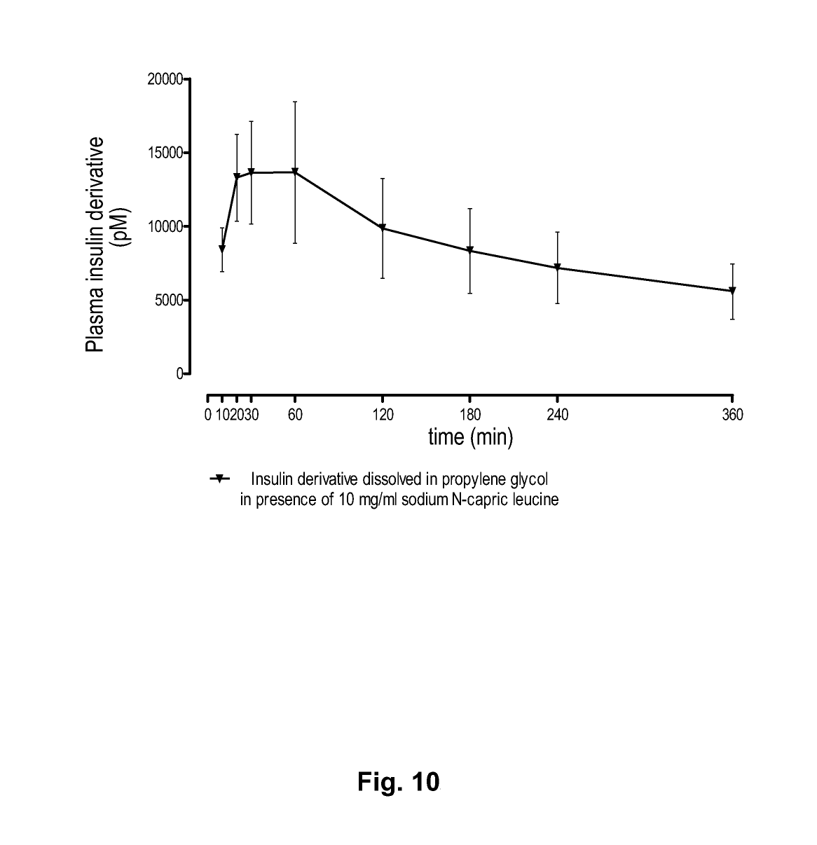

The result is shown in FIG. 10.

### Example 11

Pharmakokinetic profiles were retrieved from the resulting records related to measurements after peroral dosing of an enteric coated tablet comprising 200 mg of sodium lauroyl sarcosinate, 50 mg of soybean trypsine inhibitor (SBTI) and Eudragit® L30-D55 and Eudragit® NE30D for enteric coating further comprising insulin derivative A14E, B25H, B29K(N(eps)Octadecanedioyl-gGlu-OEG-OEG) and desB30 human insulin (120 nmol/kg) after peroral dosing of an enteric coated tablet comprising 200 mg of sodium lauroyl sarcosinate, 50 mg of soybean trypsine inhibitor (SBTI) and Eudragit® L30-D55 and Eudragit® NE30D for enteric coating to male beagle dogs.

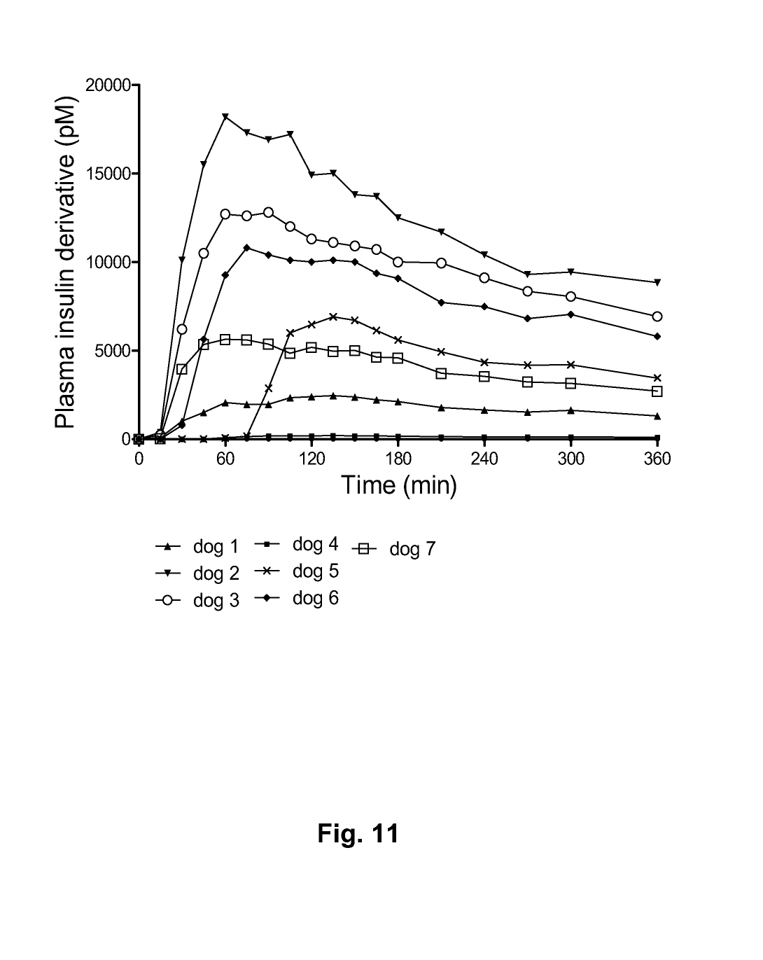

The results are shown in FIG. 11 as single PK profiles.

### Example 12

The insulin derivative A14E, B25H, B29K(N(eps)Octadecanedioyl-gGlu-OEG-OEG), desB30 human insulin (60 nmol/kg) dissolved in phosphate buffer (pH 7.4) in presence of 10 mg sodium lauroyl leucine, a mixtures of 5 mg/mL sodium lauroyl leucine and 5 mg/mL capric leucine or 10 mg/mL of the commonly used permeation enhancers salicylate, deoxycholate. The resulting compositions were injected into mid-jejunum of anaesthetized overnight fasted Sprague-Dawley rats (n=5-6) and the pharmacokinetic profiles were calculated based on the resulting records.

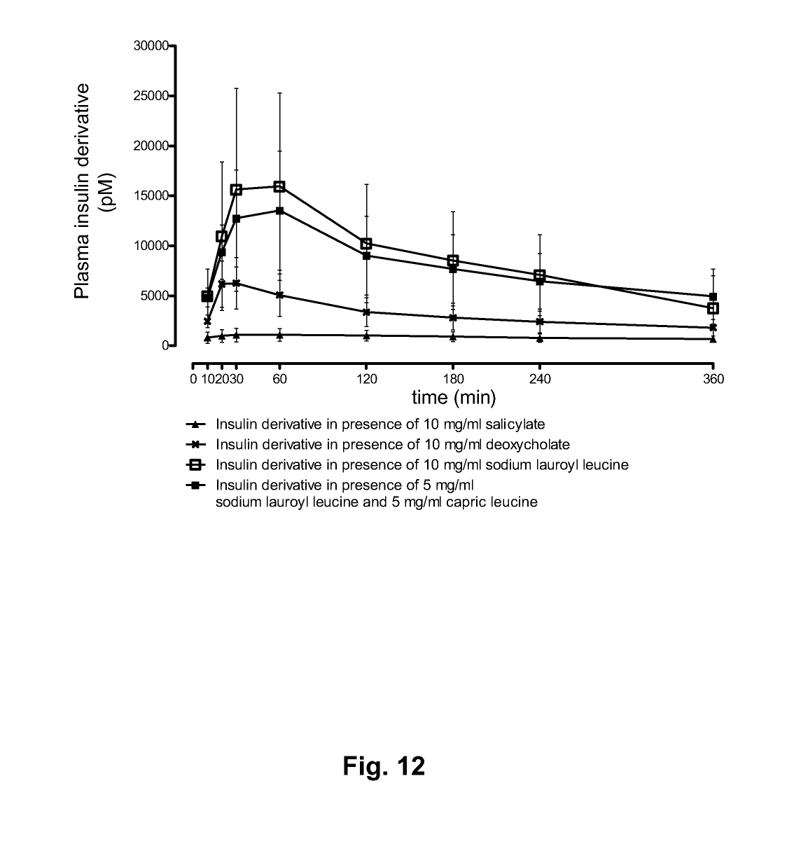

The result is shown in FIG. 12.

### Example of Liquid Non-Aqueous Pharmaceutical Compositions Comprising Insulin Derivative and Fatty Acid Acylated Amino Acids

### Example 13

Liquid insulin SEDDS, SMEDDS and SNEDDS formulations were prepared according to the guidance given in WO08145728 comprising the fatty acid acylated amino acid sodium N-lauroyl phenylalanine.

All formulations contained the insulin derivative A14E, B25H, B29K(N(eps)Octadecanedioyl-gGlu-OEG-OEG), desB30 human insulin (30 nmol/kg).

The insulin derivative A14E, B25H, B29K(N(eps)Octadecanedioyl-gGlu-OEG-OEG), desB30 human insulin (30 nmol/kg) dissolved in liquid SEDDS, SMEDDS and SNEDDS formulations comprising sodium N-lauroyl phenylalanine.

The resulting compositions were injected into mid-jejunum of anaesthetized overnight fasted Sprague-Dawley rats (n=5-6) and the pharmacokinetic profiles were calculated based on the resulting records.

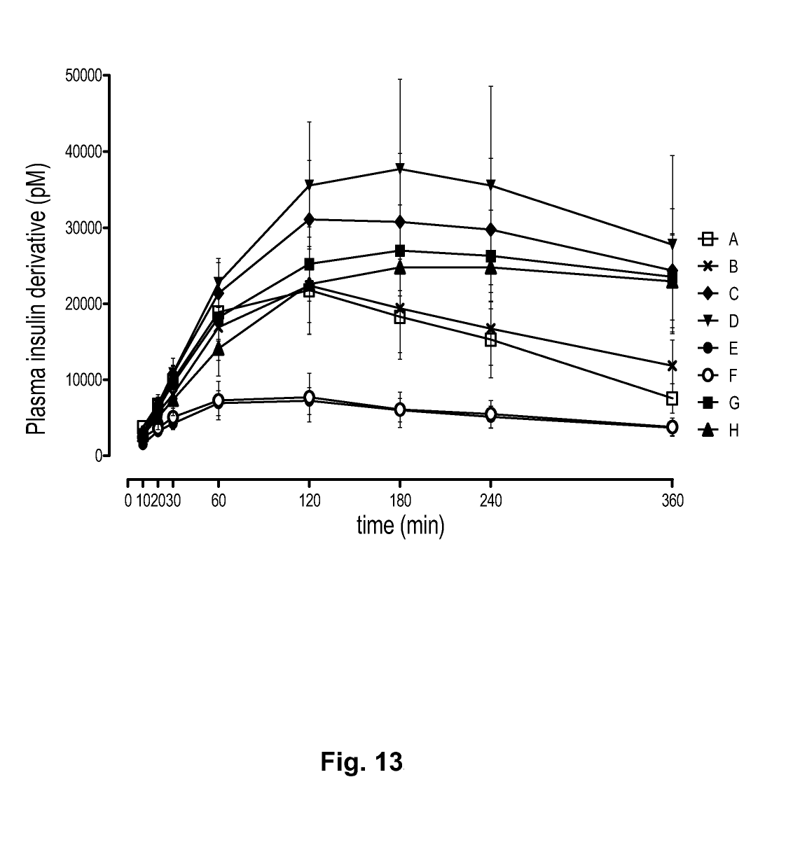

The compositions are shown in table 1 and PK results are shown in FIG. 13.

The compositions are shown in Table 1.

TABLE 1
Table 1 Liquid insulin SEDDS, SMEDDS and SNEDDS formulations
comprising the co-surfactant sodium N-lauroyl phenylalanine.
All formulations contain the insulin derivative A14E, B25H,
B29K(N(eps)Octadecanedioyl-gGlu-OEG-OEG),
desB30 human insulin (30 nmol/kg).

| | | Sodium | | |
|---|---|---|---|---|
| | Tween | N-lauroyl | Diglycerol | Propylene |
| | 20 | phenylalanine | caprylate | glycol |
| | (w/w %) | (w/w %) | (w/w %) | (w/w %) |
| A | 35 | 0 | 50 | 15 |
| B | 5 | 10 | 70 | 15 |
| C | 35 | 5 | 45 | 15 |
| D | 30 | 10 | 45 | 15 |
| E | 10 | 0 | 75 | 15 |
| F | 5 | 5 | 75 | 15 |
| G | 20 | 5 | 60 | 15 |
| H | 20 | 5 | 60 | 15 |

### Example 14

Insulin SEDDS and SMEDDS compositions were prepared according to the guidance given in WO08145728 comprising at least one fatty acid acylated amino acid (FA-aa). Mean particle size (hydrodynamic diameter) was analysed after 100 fold dilution in MilliQ water at 37° C. and respective PDI (poly dispersity index). All formulations contained the insulin derivative A14E, B25H, B29K(N(eps)Octadecanedioyl-gGlu-OEG-OEG), desB30 human insulin (30 nmol/kg).

The results are shown in Table 2.

TABLE 2
Table 2 Insulin SEDDS and SMEDDS compositions comprising at least one FA-aa.
Mean particle size (hydrodynamic diameter) is shown after 100 fold dilution in
MilliQ water at 37° C. and respective PDI (poly dispersity index).

| | | | | | Mean | | |
|---|---|---|---|---|---|---|---|
| Insulin | Propylene | FA-aa | Tween | Diglycerol | particle size | | |
| derivative | glycol | (50 mg) | 20 | caprylate | (diameter) | | PDI |
| 5 mg | 150 mg | Sodium N- | 300 mg | 500 mg | 170.2 | nm | 0.226 |
| | | capric | | | | | |
| | | serine | | | | | |
| 5 mg | 150 mg | Sodium N- | 300 mg | 500 mg | 75.7 | nm | 0.131 |
| | | capric | | | | | |
| | | histidine | | | | | |
| 5 mg | 150 mg | Sodium N- | 300 mg | 500 mg | 91.4 | nm | 0.168 |
| | | capric | | | | | |
| | | threonine | | | | | |
| 5 mg | 150 mg | Sodium N- | 300 mg | 500 mg | 151.1 | nm (74%) | 0.483 |
| | | capric | | | 32.0 | nm (27%) | |
| | | phenylalanine | | | | | |
| 5 mg | 150 mg | Sodium N- | 300 mg | 500 mg | 104.1 | nm | 0148 |
| | | capric | | | | | |
| | | aspartate | | | | | |
| 5 mg | 150 mg | Sodium N- | 300 mg | 500 mg | 110.6 | nm | 0.188 |
| | | capric | | | | | |
| | | proline | | | | | |
| 5 mg | 150 mg | Sodium N- | 300 mg | 500 mg | 209.1 | nm | 0.461 |
| | | capric | | | | | |
| | | leucine | | | | | |
| 5 mg | 150 mg | Sodium N- | 300 mg | 500 mg | 53.8 | nm (75%) | 0.442 |
| | | capric | | | 328 | nm (25%) | |
| | | isoleucine | | | | | |
| 5 mg | 150 mg | Sodium N- | 300 mg | 500 mg | 71.4 | nm | 0.130 |
| | | capric | | | | | |
| | | alanine | | | | | |
| 5 mg | 150 mg | Sodium N- | 300 mg | 500 mg | 43.1 | nm | 0.325 |
| | | capric | | | | | |
| | | tyrosine | | | | | |
| 5 mg | 150 mg | Sodium N- | 300 mg | 500 mg | 93.4 | nm (80%) | 0.482 |
| | | capric | | | 1092 | nm (20%) | |
| | | glutamine | | | | | |
| 5 mg | 150 mg | Sodium N- | 300 mg | 500 mg | 61.46 | nm | 0.157 |
| | | lauroyl | | | | | |
| | | valine | | | | | |
| 5 mg | 150 mg | Sodium N- | 300 mg | 500 mg | 30.33 | nm (72%) | 0.777 |
| | | lauroyl | | | 867 | nm (18%) | |
| | | isoleucine | | | | | |
| 5 mg | 150 mg | Sodium N- | 300 mg | 500 mg | 47.04 | nm (86%) | 0.502 |
| | | lauroyl | | | 15.23 | nm (14%) | |
| | | tyrosine | | | | | |
| 5 mg | 150 mg | Sodium N- | 300 mg | 500 mg | 119.8 | nm | 0.162 |
| | | lauroyl | | | | | |
| | | serine | | | | | |
| 5 mg | 150 mg | Sodium N- | 300 mg | 500 mg | 61.84 | nm | 0.198 |
| | | lauroyl | | | | | |
| | | glycine | | | | | |
| 5 mg | 150 mg | Sodium N- | 300 mg | 500 mg | 31.32 | nm (87%) | 0.272 |
| | | lauroyl | | | 06.36 | nm (13%) | |
| | | trypsine | | | | | |
| 5 mg | 150 mg | Sodium N- | 300 mg | 500 mg | 91.07 | nm | 0.175 |
| | | lauroyl | | | | | |
| | | alanine | | | | | |
| 5 mg | 150 mg | Sodium N- | 300 mg | 500 mg | 87.95 | nm | 0.185 |
| | | lauroyl | | | | | |
| | | histidine | | | | | |
| 5 mg | 150 mg | Sodium N- | 300 mg | 500 mg | 74.55 | nm (62%) | 0.442 |
| | | lauroyl | | | 686.6 | nm (38%) | |
| | | glutamine | | | | | |
| 5 mg | 150 mg | Sodium N- | 300 mg | 500 mg | 45.57 | nm | 0.241 |
| | | lauroyl | | | | | |
| | | aspartate | | | | | |
| 5 mg | 150 mg | Sodium N- | 300 mg | 500 mg | 102.2 | nm | 0.171 |
| | | lauroyl | | | | | |
| | | proline | | | | | |

### Example 15

Enteric softcapsule comprising insulin derivative and fatty acid acylated amino acids formulated in a SEDDS. Insulin SEDDS compositions were prepared according to the guidance given in WO08145728 (in short, the insulin was first dissolved in water and the pH adjusted to pH 7.4 with a non volatile base (NaOH) followed by freeze drying, the resulting insulin powder was then dissolved first in propylene glycol and then mixed with the other excipients as described) comprising at least one fatty acid acylated amino acid (FA-aa). Pharmakokinetic profile in a single beagle dog is shown of the insulin derivative A1(N,N-Dimethyl), A14E, B1(N,N-dimethyl), B25H, B29K(N(eps)octadecanedioyl-gGlu-OEG-OEG), desB30 human insulin (120 nmol/kg) after peroral dosing of an enteric coated soft capsule comprising 30 mg of sodium lauroyl leucine sodium salt, 150 mg of propylene glycol, 300 mg of Polysorbate 20 and 520 mg of diglycerol monocaprylate.

Softcapsule was enteric coated with a mixture of Eudragit® L30-D55 and Eudragit® NE30D.

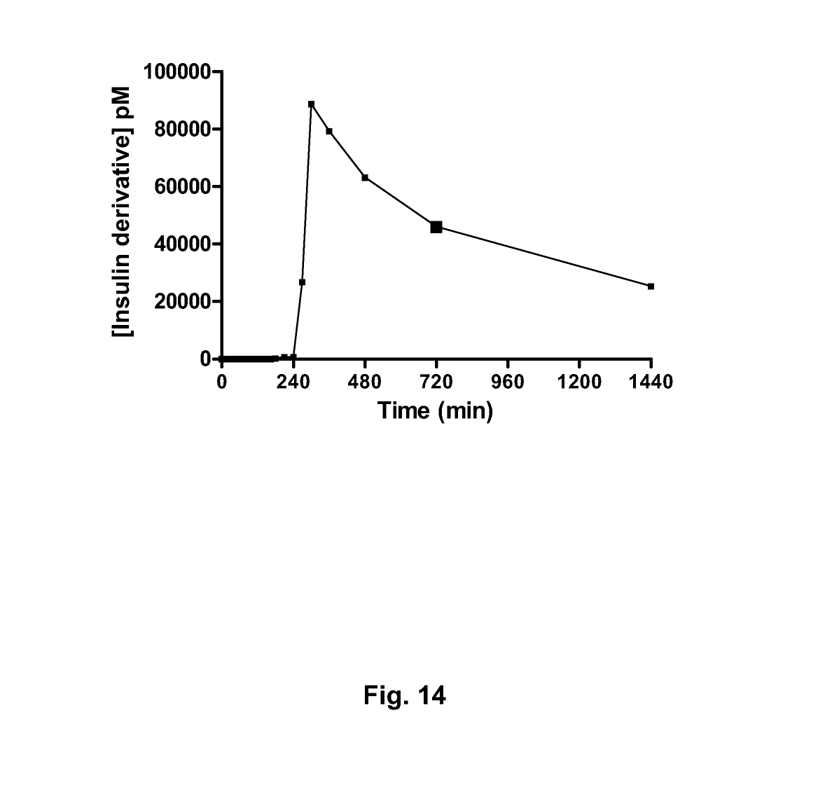

The result is shown in FIG. 14 as single PK profile.

### Example 16

Liquid non-aqueous insulin analogue compositions with different amounts of N-lauroyl leucine sodium salt. Insulin SEDDS compositions were prepared according to the guidance given in WO08145728 (in short, the insulin was first dissolved in water and the pH adjusted to pH 7.4 with a non volatile base (NaOH) followed by freeze drying, the resulting insulin powder was then dissolved first in propylene glycol and then mixed with the other excipients as described) comprising at least one fatty acid acylated amino acid (FA-aa). Insulin SEDDS and SMEDDS compositions were prepared comprising increasing amounts of N-lauroyl leucine sodium salt. Mean particle size (hydrodynamic diameter) was analysed after 50 fold dilution in MilliQ water at 37° C. and respective PDI (poly dispersity index). All formulations contained the insulin derivative A14E, B25H, B29K(N(eps)Octadecanedioyl-gGlu-OEG-OEG), desB30 human insulin (5 mg/g).

The results are shown in Table 3.

TABLE 3
Table 3. Liquid insulin analogue compositions comprising
different amounts of N-lauroyl leucine sodium salt.

| | | | | | Particle size | |
|---|---|---|---|---|---|---|
| | | | | | (50 fold dilution | |
| | Pro- | Poly- | Diglycerol | N-lauroyl | in MilliQ water) | |
| | pylene | sorbate | mono- | leucine | Z average | |
| NO | glycol | 20 | caprylate | sodium salt | (nm) | PDI |
| 1 | 15% | 30% | 52% | 3% | 49 | 0.126 |
| 2 | 15% | 30% | 50% | 5% | 66 | 0.2 |
| 3 | 15% | 30% | 48% | 7% | 1866 | 1 |
| 4 | 15% | 30% | 40% | 10% | 282 | 0.6 |

### Example 17

Liquid insulin analogue compositions with different amounts of N-lauroyl leucine sodium salt further comprising diglycerol monocaprylate and propylene glycol. Insulin SEDDS compositions were prepared according to the guidance given in WO08145728 (in short, the insulin was first dissolved in water and the pH adjusted to pH 7.4 with a non volatile base (NaOH) followed by freeze drying, the resulting insulin powder was then dissolved first in propylene glycol and then mixed with the other excipients as described) comprising at least one fatty acid acylated amino acid (FA-aa). Insulin SEDDS compositions were prepared comprising different amounts of N-lauroyl leucine sodium salt. Mean particle size (hydrodynamic diameter) was analysed after 50 fold dilution in MilliQ water at 37° C. and respective PDI (poly dispersity index). All formulations contained the insulin derivative A14E, B25H, B29K(N(eps)Octadecanedioyl-gGlu-OEG-OEG), desB30 human insulin (5 mg/g).

The results are shown in Table 4.

TABLE 4
Table 4
Liquid insulin analogue compositions with different amounts of
N-lauroyl leucine sodium salt just comprising diglycerol
monocaprylate and propylene glycol.

| | | | N-lauroyl | | |
|---|---|---|---|---|---|
| | | Diglycerol | leucine | Particle size | |
| | Propylene | mono | sodium | (1:10 dilution mQ water) | |
| No | glycol % | caprylate % | salt % | Z average (nm) | PDI |
| 1 | 15% | 80 | 5 | 7.3 | 0.226 |
| 2 | 15% | 77.5 | 7.5 | 5.5 | 0.23 |
| 3 | 15% | 75 | 10 | 223 | 0.255 |
| 4 | 15% | 72.5 | 12.5 | 44 | 0.281 |
| 5 | 15% | 70 | 15 | 309 | 0.384 |
| 6 | 15% | 67.5 | 17.5 | 330 | 0.35 |
| 7 | 15% | 65 | 20 | 769 | 0.699 |
| 8 | 15% | 62.5 | 22.5 | 728 | 0.607 |
| 9 | 15% | 60 | 25 | 642 | 0.629 |
| 10 | 15% | 57.5 | 27.5 | 352 | 0.359 |

### Example 18

Lipid compositions with different fatty acid acylated aminoacids, various solvents and different Polysorbate. Insulin SEDDS compositions were prepared according to the guidance given in WO08145728 (in short, the insulin was first dissolved in water and the pH adjusted to pH 7.4 with a non volatile base (NaOH) followed by freeze drying, the resulting insulin powder was then dissolved first in propylene glycol and then mixed with the other excipients as described) comprising at least one fatty acid acylated amino acid (FA-aa). Insulin SEDDS and SMEDDS compositions were prepared comprising different fatty acid acylated amino acid sodium salts, polysorbates and solvents. Mean particle size (hydrodynamic diameter) was analysed after 50 fold dilution in MilliQ water at 37° C. and respective PDI (poly dispersity index). All formulations comprise 5 mg/g insulin analogue A1(N,N-Dimethyl), A14E, B1(N,N-dimethyl), B25H, B29K(N(eps)octadecanedioyl-gGlu-OEG-OEG), desB30 human insulin 5 mg/g. The results are shown in Table 5.

TABLE 5
Table 5. Lipid compositions with different fatty acid acylated
aminoacids, various solvents and different Polysorbates.

| | | | | | Particle size (1:50 | |
|---|---|---|---|---|---|---|
| | | Fatty acid | | Diglycerol | MilliQ water) | |
| | | acylated | | mono- | Z | |
| | Solvent | aminoacid | Surfactant | caprylate | average | |
| No | 15% | 5% | 35% | (45%) | (nm) | PDI |
| 1 | Propylene | N-lauroyl | Tween 20 | Diglycerol | 570 | 0.52 |
| | glycol | leucine | | mono- | | |
| | | sodium salt | | caprylate | | |
| 2 | Propylene | N-lauroyl | Tween 40 | Diglycerol | 268 | 0.35 |
| | glycol | leucine | | mono- | | |
| | | sodium salt | | caprylate | | |
| 3 | Propylene | N-lauroyl | Tween 60 | Diglycerol | 97 | 0.128 |
| | glycol | leucine | | mono- | | |
| | | sodium salt | | caprylate | | |
| 4 | Propylene | N-lauroyl | Tween 80 | Diglycerol | 115 | 0.185 |
| | glycol | leucine | | mono- | | |
| | | sodium salt | | caprylate | | |
| 5 | Propylene | N-capric | Tween 20 | Diglycerol | 74 | 0.53 |
| | glycol | leucine | | mono- | | |
| | | sodium salt | | caprylate | | |
| 6 | Propylene | N-capric | Tween 40 | Diglycerol | 87 | 0.56 |
| | glycol | leucine | | mono- | | |
| | | sodium salt | | caprylate | | |
| 7 | Propylene | N-capric | Tween 60 | Diglycerol | 12 | 0.29 |
| | glycol | leucine | | mono- | | |
| | | sodium salt | | caprylate | | |
| 8 | Propylene | N-capric | Tween 80 | Diglycerol | 125 | 0.33 |
| | glycol | leucine | | mono- | | |
| | | sodium salt | | caprylate | | |
| 9 | Propylene | N-lauroyl | Tween 20 | Diglycerol | 7.5 | 0.24 |
| | glycol | sarcosinate | | mono- | | |
| | | | | caprylate | | |
| 10 | Propylene | N-lauroyl | Tween 40 | Diglycerol | 7.5 | 0.26 |
| | glycol | sarcosinate | | mono- | | |
| | | | | caprylate | | |
| 11 | Propylene | N-lauroyl | Tween 60 | Diglycerol | 7.4 | 0.27 |
| | glycol | sarcosinate | | mono- | | |
| | | | | caprylate | | |
| 12 | Propylene | N-lauroyl | Tween 80 | Diglycerol | 14.7 | 0.41 |
| | glycol | sarcosinate | | mono- | | |
| | | | | caprylate | | |
| 13 | H2O | N-lauroyl | Tween 20 | Diglycerol | 178 | 0.25 |
| | | leucine | | mono- | | |
| | | sodium salt | | caprylate | | |
| 14 | H2O | N-lauroyl | Tween 40 | Diglycerol | 740 | 0.55 |
| | | leucine | | mono- | | |
| | | sodium salt | | caprylate | | |
| 15 | H2O | N-lauroyl | Tween 60 | Diglycerol | 196 | 0.29 |
| | | leucine | | mono- | | |
| | | sodium salt | | caprylate | | |
| 16 | H2O | N-lauroyl | Tween 80 | Diglycerol | 170 | 0.26 |
| | | leucine | | mono- | | |
| | | sodium salt | | caprylate | | |
| 17 | H2O | N-lauroyl | Tween 20 | Diglycerol | 8 | 0.27 |
| | | sarcosinate | | mono- | | |
| | | | | caprylate | | |
| 18 | H2O | N-lauroyl | Tween 40 | Diglycerol | 8 | 0.27 |
| | | sarcosinate | | mono- | | |
| | | | | caprylate | | |
| 19 | H2O | N-lauroyl | Tween 60 | Diglycerol | 10 | 0.34 |
| | | sarcosinate | | mono- | | |
| | | | | caprylate | | |
| 20 | H2O | N-lauroyl | Tween 80 | Diglycerol | 17.7 | 0.47 |
| | | sarcosinate | | mono- | | |
| | | | | caprylate | | |

### Example 19

Liquid lipid based formulations comprising at least one fatty acid acylated amino acid, insulin derivative, solvent and at least one lipid or co-surfactant were prepared. Insulin SEDDS compositions were prepared according to the guidance given in WO08145728 (in short, the insulin was first dissolved in water and the pH adjusted to pH 7.4 with a non volatile base (NaOH) followed by freeze drying, the resulting insulin powder was then dissolved first in propylene glycol and then mixed with the other excipients as described) comprising at least one fatty acid acylated amino acid (FA-aa).

Insulin SEDDS compositions were prepared comprising different fatty acid acylated amino acid sodium salts, lipid or co-surfactant and a solvent. Mean particle size (hydrodynamic diameter) was analysed after 50 fold dilution in MilliQ water at 37° C. and respective PDI (poly dispersity index). All formulations comprise 5 mg/g insulin analogue A1(N,N-Dimethyl), A14E, B1(N,N-dimethyl), B25H, B29K(N(eps)octadecanedioyl-gGlu-OEG-OEG), desB30 human insulin. The results are shown in Table 6.

TABLE 6
Table 6
Liquid lipid based formulations comprising at least
one fatty acid acylated aminoacid, insulin derivative,
solvent and at least one lipid or co-surfactant are described.

| | | Fatty acid | |
|---|---|---|---|
| | | acylated amino | |
| | Solvent 15% | acid | Lipid or co-surfactant |
| No | (w/w %) | 5% (w/w %) | 80% (w/w %) |
| 1 | Propylene | N-lauroyl leucine | Diglycerol mono caprylate |
| | glycol | sodium salt | |
| 2 | Propylene | N-lauroyl leucine | Glycerol mono caprylate |
| | glycol | sodium salt | |
| 3 | Propylene | N-lauroyl sarcosinate | Diglycerol mono caprylate |
| | glycol | | |
| 4 | Propylene | N-lauroyl sarcosinate | Glycerol mono caprylate |
| | glycol | | |
| 5 | H2O | N-lauroyl leucine | Diglycerol mono caprylate |
| | | sodium salt | |
| 6 | H2O | N-lauroyl leucine | Glycerol mono caprylate |
| | | sodium salt | |
| 7 | H2O | N-lauroyl sarcosinate | Diglycerol mono caprylate |
| 8 | H2O | N-lauroyl sarcosinate | Glycerol mono caprylate |

### Example 20

Lipid SEDDS, SMEDDS and SNEDDS compositions comprising N-lauroyl leucine sodium salt and different surfactants with variable HLB values were prepared. Insulin SEDDS compositions were prepared according to the guidance given in WO08145728 comprising at least one fatty acid acylated amino acid (FA-aa).

Insulin SEDDS and SMEDDS compositions were prepared comprising N-lauroyl leucine sodium salt, propylene glycol, diglycerol mono caprylate and a high or low HLB surfactant. All formulations comprise 5 mg/g insulin analogue A1(N,N-Dimethyl), A14E, B1(N,N-dimethyl), B25H, B29K(N(eps)octadecanedioyl-gGlu-OEG-OEG), desB30 human insulin.

Mean particle size (hydrodynamic diameter) was analysed after 50 fold dilution in MilliQ water at 37° C. and respective PDI (poly dispersity index). All formulations comprise 5 mg/g insulin analogue A1(N,N-Dimethyl), A14E, B1(N,N-dimethyl), B25H, B29K(N(eps)octadecanedioyl-gGlu-OEG-OEG), desB30 human insulin. The results are shown in Table 7.

The lipid compositions analysed and shown in table 7 were composed as follows;

| | |
|---|---|
| Insulin derivative (constant) | 5 mg/ml |
| Propylene glycol (constant) | 15% |
| Diglycerol mono caprylate | 55% |
| (constant) | |
| N-lauroyl leucine sodium salt | 5% |
| (constant) | |
| Surfactant or co-surfactant (see | Up to 25% |
| Table 7) | |

TABLE 7
Table 7. Lipid compositions comprising N-lauroyl leucine sodium
salt and different surfactants with variable HLB values.

| | | | | Visual | | |
|---|---|---|---|---|---|---|
| | | | | appear- | | |
| | Surfactant | | | ance | (Z) | |
| | or co- | | Formulation | after | average | |
| NO | surfactant | HLB | appearance | dilution | (nm) | PDI |
| 1 | Span 80 | 4.3 | clear liquid | clear | 35 | 0.116 |
| | | | | solution | | |
| 2 | Span 60 | 4.8 | insoluble | X | X | X |
| 3 | Span 40 | 6.7 | clear liquid | turbid | 2767 | 0.36 |
| | | | with | solution | | |
| | | | heating | | | |
| 4 | Span 20 | 8.6 | clear liquid | clear | 48 | 0.44 |
| | | | | solution | | |
| 5 | Span 10 | 9.4 | insoluble | X | X | X |
| 6 | Span 8 | 10.3 | little | clear | 4048 | 1.0 |
| | | | turbid | solution | | |
| 7 | Span 6 | 11.4 | clear liquid | clear | 5436 | 1.0 |
| | | | | solution | | |
| 8 | Tween 81 | 10 | clear liquid | clear | 60 | 0.18 |
| | | | | solution | | |
| 9 | Tween 65 | 10.5 | clear liquid | clear | 6 | 0.294 |
| | | | with | solution | | |
| | | | heating | | | |
| 10 | Tween 85 | 11 | clear liquid | clear | 12 | 0.37 |
| | | | | solution | | |
| 11 | Tween 21 | 13.3 | clear liquid | clear | 93 | 0.22 |
| | | | | solution | | |
| 12 | Tween 60 | 14.8 | clear liquid | clear | 1.7 | 0.16 |
| | | | | solution | | |
| 13 | Tween 80 | 15 | clear liquid | clear | 330 | 0.56 |
| | | | | solution | | |
| 14 | Tween 40 | 15.6 | clear liquid | clear | 615 | 0.83 |
| | | | | solution | | |
| 15 | Tween 20 | 16.7 | clear liquid | clear | 751 | 1.0 |
| | | | | solution | | |
| 16 | Poloxamer124 | dec-18 | clear liquid | clear | 185 | 0.27 |
| | | | | solution | | |
| 17 | Deoxy- | 16 | clear liquid | clear | 3012 | 1.0 |
| | cholate Na | | | solution | | |
| 18 | Tauro- | N/A | clear liquid | clear | 1673 | 1.0 |
| | cholate Na | | | solution | | |

### Example of Other Compositions

### Example 21

The composition of the insulin degludec/liraglutide drug product that Novo Nordisk A/S currently has in clinical development is shown below. This formulation has been shown to be a stable combination product suitable for use in type II diabetes clinical trials (subcutaneous injection).”

### Names of Ingredients in the Drug Product Formulation

Drug substances
Liraglutide, 3.6 mg (960 nmol) per ml
Insulin degludec, 600 nmol (100 U) per ml

### Excipients

Phenol
Glycerol
Zinc

### Formulation Process Specialties

Both insulin degludec and liraglutide drug substances are added in the form of a solid powder, separately and directly to a mixture of excipients.
All of the zinc is added in one step.
There is no need for holding time anywhere in the formulation process.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An oral pharmaceutical composition comprising
a. at least one fatty acid acylated amino acid of the general formula: 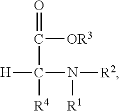
wherein R1 is a fatty acid chain comprising 8 to 18 carbon atoms,
R2 is either H (i.e. hydrogen) or CH3 (i.e. methyl group), and
R3 is either H, or a salt thereof, and
R4 is a non-cationic amino acid side chain, and
b. at least one hydrophillic peptide or protein.

2. The pharmaceutical composition according to claim 1, wherein said hydrophilic peptide or protein is an insulin peptide.

3. The pharmaceutical composition according to claim 1, which comprises less than 10% (w/w) water.

4. The oral pharmaceutical composition according to claim 1, wherein the amino acid residue of said at least one fatty acid acylated amino acid is based on a nonpolar hydrophobic amino acid, a polar uncharged amino acid or polar acidic amino acid.

5. The oral composition according to claim 1 further comprising an enteric or delayed release coating.

6. The oral pharmaceutical composition according to claim 1, wherein the fatty acid acylated amino acid is in the form of its free acid or salt.

7. The oral pharmaceutical composition according to claim 1 wherein said fatty acid moiety of the fatty acid acylated amino acid consists of 10, 12, 14, 16 or 18 carbon atoms.

8. The oral pharmaceutical composition according to claim 1, wherein the amino acid residue of said fatty acid acylated amino acid is selected from the group consisting of Alanine (Ala), Valine (Val), Leucine (Leu), Isoleucine (Ile), Phenylalanine (Phe), Tryptophane (Trp), Metheonine (Met), Proline (Pro), Sarcosinate, Glycine (Gly), Serine (Ser), Threonine (Thr), Cysteine (Cys), Tyrosine (Tyr), Asparagine (Apn), and Glutamine (Gln), Aspartic acid (Asp) and Glutamic acid (Glu).

9. The oral pharmaceutical composition according to claim 1, wherein the fatty acid acylated amino acid is selected from the group consisting of: Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine, Sodium cocoyl glycine, Sodium cocoyl glutamate Sodium lauroyl alaninate, N-dodecanoyl-L-alanine, Sodium lauroyl asparaginate, N-dodecanoyl-L-asparagine, Sodium lauroyl aspartic acid, N-dodecanoyl-L-aspartic acid, Sodium lauroyl cysteinate, N-dodecanoyl-L-cysteine, Sodium lauroyl glutamic acid, N-dodecanoyl-L-glutamic acid, Sodium lauroyl glutaminate, N-dodecanoyl-L-glutamine, Sodium lauroyl glycinate, N-dodecanoyl-L-glycine, Sodium lauroyl histidinate, N-dodecanoyl-L-histidine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl serinate, N-dodecanoyl-L-serine, Sodium lauroyl threoninate, N-dodecanoyl-L-threonine, Sodium lauroyl tryptophanate, N-dodecanoyl-L-tryptophane, Sodium lauroyl tyrosinate, N-dodecanoyl-L-tyrosine, Sodium lauroyl valinate, N-dodecanoyl-L-valine, Sodium lauroyl sarcosinate, N-dodecanoyl-L-sarcosine, Sodium capric alaninate, N-decanoyl-L-alanine, Sodium capric asparaginate, N-decanoyl-L-asparagine, Sodium capric aspartic acid, N-decanoyl-L-aspartic acid, Sodium capric cysteinate, N-decanoyl-L-cysteine, Sodium capric glutamic acid, N-decanoyl-L-glutamic acid, Sodium capric glutaminate, N-decanoyl-L-glutamine, Sodium capric glycinate, N-decanoyl-L-glycine, Sodium capric histidinate, N-decanoyl-L-histidine, Sodium capric isoleucinate, N-decanoyl-L-isoleucine, Sodium capric leucinate, N-decanoyl-L-leucine, Sodium capric methioninate, N-decanoyl-L-methionine, Sodium capric phenylalaninate, N-decanoyl-L-phenylalanine, Sodium capric prolinate, N-decanoyl-L-proline, Sodium capric serinate, N-decanoyl-L-serine, Sodium capric threoninate, N-decanoyl-L-threonine, Sodium capric tryptophanate, N-decanoyl-L-tryptophane, Sodium capric tyrosinate, N-decanoyl-L-tyrosine, Sodium capric valinate, N-decanoyl-L-valine, Sodium capric sarcosinate and N-decanoyl-L-sarcosine, Sodium lauroyl sarcosinate, Sodium oleoyl sarcosinate, Sodium N-decyl leucine, Amisoft HS-11 P (Sodium Stearoyl Glutamate, Amisoft MS-11 (Sodium Myristoyl Glutamate)), Amisoft LS-11 (Sodium Lauroyl Glutamate), Amisoft CS-11 (Sodium Cocoyl Glutamate), Amilite GCS-11 (Sodium Cocoyl Glycinate), Sodium lauroyl sarcosinate, Sodium N-decyl leucine and Sodium cocoyl glycine, Sodium cocoyl glutamate.

10. The oral pharmaceutical composition according to claim 9, further comprising propylene glycol.

11. An oral pharmaceutical composition according to claim 9, further comprising SEDDS, SMEDDS or SNEDDS.

12. The oral pharmaceutical composition according to claim 9, further comprising other pharmaceutical excipients.

13. (canceled)

14. (canceled)

15. (canceled)

16. A method for treating diabetes mellitus in a subject in need of such treatment, said method comprising administering to said subject a therapeutically effective amount of the oral pharmaceutical composition of claim 1.

17. A method for treating diabetes mellitus in a subject in need of such treatment, said method comprising administering to said subject a therapeutically effective amount of the oral pharmaceutical composition of claim 9.

18. The oral pharmaceutical composition according to claim 1, further comprising propylene glycol.

19. An oral pharmaceutical composition according to claim 18, further comprising SEDDS, SMEDDS or SNEDDS.
